(19) United States
(12) Patent Application Publication
(10) Pub. No.: US 2022/0051798 A1
(43) Pub. Date: Feb. 17, 2022

(54) System, Method and Computer Readable Medium for Modeling Biobehavioral Rhythms from Mobile and Wearable Data Streams

(71) Applicant: University of Virginia Patent Foundation, Charlottesville, VA (US)

(72) Inventors: Afsaneh Doryab, Charlottesville, VA (US); Runze Yan, Charlottesville, VA (US); Xinwen Liu, Charlottesville, VA (US)

(73) Assignee: University of Virginia Patent Foundation, Charlottesville, VA (US)

(21) Appl. No.: 17/398,097

(22) Filed: Aug. 10, 2021

Related U.S. Application Data
(60) Provisional application No. 63/064,075, filed on Aug. 11, 2020.
(60) Provisional application No. 63/230,496, filed on Aug. 6, 2021.

(51) Int. Cl. G16H 50/20; G06F 30/27; G16H 50/30; G16H 40/67; G16H 50/70; G01N 33/483; A61B 5/00

(52) U.S. Cl. CPC G16H 50/20; G06F 30/27; G16H 50/30; G06F 2111/10; G16H 50/70; G01N 33/483; A61B 5/4857; G16H 40/67

(57) ABSTRACT

A technique for providing biobehavioral rhythm models that generate a series of characteristic features which are further used for measuring stability in biobehavioral rhythms and to predict different outcomes such as health status through a machine learning component. A computational framework is provided for modeling biobehavioral rhythms from mobile and wearable data streams that rigorously processes sensor streams, detects periodicity in data, models rhythms from that data and uses the cyclic model parameters to predict an outcome. The framework can reliably discover various periods of different length in data, extract cyclic biobehavioral characteristics through exhaustive modeling of rhythms for each sensor feature; and provide the ability to use different combination of sensors and data features to predict an outcome.

### CROSS REFERENCE TO RELATED APPLICATIONS

The present application claims benefit of priority under 35 U.S.C § 119 (e) from U.S. Provisional Application Ser. No. 63/064,075, filed Aug. 11, 2020, entitled “System and Method for Modeling Biobehavioral Rhythms from Mobile and Wearable Data Streams” and U.S. Provisional Application Ser. No. 63/230,496, filed Aug. 6, 2021, entitled “System and Method for Modeling Biobehavioral Rhythms from Mobile and Wearable Data Streams”; the disclosures of which are hereby incorporated by reference herein in their entirety.

### STATEMENT OF GOVERNMENT INTEREST

This invention was made with government support under Grant No. 2023762, awarded by the National Science Foundation. The government has certain rights in the invention.

### FIELD OF INVENTION

The present disclosure relates generally to modeling biobehavioral rhythms of a subject. More particularly, the present disclosure relates to generating biobehavioral rhythm models that provide a series of characteristic features which are further used for measuring stability in biobehavioral rhythms and to predict different outcomes such as health status through a machine learning component.

### BACKGROUND

Introduction. The term biobehavioral rhythms introduced in [18], refers to the repeating cycles of physiological (e.g., heart rate and body temperature), psychological (e.g., mood), social (e.g., work events), and environmental (e.g., weather) that affect human body and life. Rooted in Chronobiology, “the scientific discipline that quantifies and explores the mechanisms of biological time structure and their relationship to the rhythmic manifestations in living matter” [14], biobehavioral rhythms aim at studying cyclic events observed in human data collected from personal and consumer level mobile and wearable devices [18]. Such devices provide the capability of continuous tracking of biobehavioral signals of individuals in their daily life and outside of controlled lab settings which has been the standard method for studying biological rhythms.

Numerous research studies have shown the impact of understanding rhythms and their effect on human life and wellbeing. For example studies in [18, 27, 29] demonstrate the association between long-term disruption in biological rhythms and health outcomes such as cancer, diabetes, and depression. Other studies have shown the impact of shift work on quality of life in shift workers such as nurses and doctors [32, 36]. These studies, however, have often been limited to controlled settings to observe certain behaviors and effects. With passive sensing of physiological and behavioral signals from mobile and wearable devices, it is now possible to study human rhythms more broadly and holistically in the wild through collection of biobehavioral data from different sources. This opportunity, however, introduces new challenges. First, the longitudinal timeseries data collected from personal devices is massive, noisy, and incomplete requiring careful processing to extract and preserve useful fine-grained knowledge from data in various temporal granularity levels to be used for further modeling. Second, the fact that each data source (e.g., smartphone sensors) can capture different aspects of human rhythms (biological, behavioral or both) requires exploration and incorporation of each signal to identify biological and behavioral indicators on the micro and macro level that may reveal a cyclic behavior. This process can be exhaustive and needs automation. Moreover, although the modeled rhythms by themselves can provide useful insights into human health and life, the exhaustive number of rhythm models generated by each source makes it difficult for manual interpretation of the models by researchers or experts. Therefore, the present inventor herein submits that a further computational step should, among other things, incorporate those models to provide further insights into different health and lifestyle outcomes both physical and mental.

Biological Rhythms. The assessment of rhythmic phenomena in living organisms reveals the existence of events and behavior that repeat themselves in certain cycles and can be modeled with periodic functions [14, 52]. Each periodic function is specified by its average level, oscillation degree, and time of oscillation optimal. Biological rhythms, including patterns of activity and rest or circadian rhythms have been extensively studied in Chronobiology and medicine [18, 27, 29] mostly in controlled environmental settings.

The advancements in activity trackers has made it possible to study these phenomena outside of the labs and has demonstrated the reliability of such devices in capturing circadian disruptions including sleep and physical and mental health conditions. For example, studies using research grade actigraphy devices have shown differences in circadian rhythms among patients with bipolar disorder, ADHD, and schizophrenia [48]. Other studies have used the same type of data to explore circadian disruption in cancer patients undergoing chemotherapy [48]. Commercial devices such as Fitbits are now able to infer sleep duration and quality reasonably accurately. Two brief studies with healthy young adults have used activity data from Fitbit devices to quantify rest-activity rhythms and found that rhythm measurement compared well relative to research-grade actigraphy [5, 37]. Studies in [62] and [41] have also explored the capability of personal tracking devices to measure sleep compared to gold standards such as polysomnography.

Behavior Modeling in the Wild via Mobile Sensing. The study of biobehavioral rhythms also relates to research in understanding human behavior from passive sensing data collected via smartphones and wearable devices. Only few studies have actually used mobile data for understanding the circadian behavior of different chronotypes (e.g., [1-3]). Abdullah et al. [1] analyzed patterns of phone usage to demonstrate differences in the sleep behavior of early and late chronotypes. In a similar study using the same type of data, they showed the capability of using mobile data to explore daily cognition and alertness [2, 3] and found that body clock, sleep duration, and coffee intake impact alertness cycles.

Data from smartphones and wearable devices has extensively been used for modeling daily behavior patterns such as movement [16], sleep [44], and physical and social activities [46] to understand their associations with health and wellbeing. For example, Medan et al. [40] found that decreases in call, SMS messaging, Bluetooth-detected contacts, and location entropy (a measure of popularity of various places) were associated with greater depression. Wang et al. [61] monitored 48 students' behavior data for one semester and demonstrated significant correlations between data from smartphones and students' mental health and educational performance. In addition, Saeb at al [54] extracted features from GPS location and phone usage data and applied a correlation analysis to capture relationships between features and level of depression. They find that circadian movement (regularity of the 24 h cycle of GPS change), normalized entropy (mobility between favorite locations), location variance (GPS mobility independent of location), phone usage features, usage duration, and usage frequency were highly correlated with the depression score. Doryab et al. [19] studied loneliness detection through data mining and machine learning modeling of students' behavior from smartphone and Fitbit data and showed different patterns of behavior related to loneliness including less time spent off campus and in different academic facilities as well as less socialization during evening hours on weekdays among students with high level of loneliness.

Recent tools such as Rhythomic [28] and ARGUS [30] use visualization to analyze human behavior. Rhythomic is an open source R framework tool for general modeling of human behavior including circadian rhythms. ARGUS, on the other hand, focuses on visual modeling of deviations in circadian rhythms and measures their degree of irregularity. Through multiple visualization panes, the tool facilitates understanding of behavioral rhythms. This work is related to our computational framework for modeling human rhythms. However, in addition to the underlying assumption of, and a focus on, circadian rhythms only, these tools primarily enable understanding of rhythms through visualization whereas in in the present inventor's framework, an aspect of an embodiment of the present invention provides, among other things, means for processing different data sources, extracting information from them and discovering and modeling rhythms for each biobehavioral signal with different periods other than 24 hours. An aspect of an embodiment of the present invention provides, among other things, the first computational framework to extract and incorporate the parameters obtained from rhythm models in a machine learning pipeline to predict different outcomes.

There is therefore a need in the art for a better mode to predict different outcomes such as health status through a machine learning component.

There is therefore a need in the art for an effective means for processing different data sources, extracting information from them and discovering and modeling rhythms for each biobehavioral signal with different periods other than 24 hours.

There is therefore a need in the art for a means for generating new knowledge and findings through rigorous micro- and macro-level modeling of human rhythms from mobile and wearable data streams collected in the wild and using them to assess and predict different life and health outcomes.

### SUMMARY OF ASPECTS OF EMBODIMENTS OF THE PRESENT INVENTION

An aspect of an embodiment of the present invention provides, among other things, the first computational framework for modeling biobehavioral rhythms—the repeating cycles of physiological, psychological, social, and environmental events—from mobile and wearable data streams. The framework incorporates, but not limited thereto, four main components: mobile data processing, rhythm discovery, rhythm modeling, and machine learning. We evaluate the framework with two case studies using datasets of smartphone, Fitbit, and OURA smart ring to evaluate the framework's ability to 1) detect cyclic biobehavior, 2) model commonality and differences in rhythms of human participants in the sample datasets, and 3) predict their health and readiness status using models of biobehavioral rhythms. Our evaluation demonstrates the framework's ability to generate new knowledge and findings through rigorous micro- and macro-level modeling of human rhythms from mobile and wearable data streams collected in the wild and using them to assess and predict different life and health outcomes.

An aspect of an embodiment of the present invention provides a system, method and computer readable medium for, among other things, providing further insights into different health and lifestyle outcomes both physical and mental.

An aspect of an embodiment of the present invention provides a system, method and computer readable medium for, among other things, providing means for processing different data sources, extracting information from them and discovering and modeling rhythms for each biobehavioral signal with different periods other than 24 hours.

An aspect of an embodiment of the present invention provides a system, method and computer readable medium for, among other things, the framework's ability to generate new knowledge and findings through rigorous micro- and macro-level modeling of human rhythms from mobile and wearable data streams collected in the wild and using them to assess and predict different life and health outcomes.

An aspect of an embodiment of the present invention provides a system, method and computer readable medium for, among other things, biobehavioral rhythm models that provide a series of characteristic features which are further used for measuring stability in biobehavioral rhythms and to predict different outcomes such as health status through a machine learning component.

An aspect of an embodiment of the present invention provides a system, method and computer readable medium for, among other things, a computational framework for modeling biobehavioral rhythms from mobile and wearable data streams that rigorously processes sensor streams, detects periodicity in data, models rhythms from that data and uses the cyclic model parameters to predict an outcome.

An aspect of an embodiment of the present invention provides a system, method and computer readable medium for, among other things, a framework that can reliably discover various periods of different length in data, extract cyclic biobehavioral characteristics through exhaustive modeling of rhythms for each sensor feature; and provide the ability to use different combinations of sensors and data features to predict an outcome.

An aspect of an embodiment of the present invention provides a system, method and computer readable medium for, among other things, the machine learning analyses for predicting mental health and readiness demonstrated the ability of our framework to process a massive number of data streams to build and analyze micro-rhythmic models for each sensor feature and combinations of features and highlighted dominant rhythmic features for prediction of the outcome of interest.

An aspect of an embodiment of the present invention provides, among other things, a computational framework to address the aforementioned challenges through a series of data processing and modeling steps. The framework first processes the raw sensor data collected from mobile and wearable devices to extract high level features from those data streams. It then models biobehavioral rhythms for each sensor feature alone and in combination with other features to discover rhythmicity and other characteristics of cyclic behavior in the data. The biobehavioral rhythm models provide a series of characteristic features which are further used for measuring stability in biobehavioral rhythms and to predict different outcomes such as health status through a machine learning component. We evaluate the framework with two case studies. The first study uses mobile and Fitbit data collected from 138 college students over a semester to test the framework's ability to detect rhythmicity in students' data in different time frames over the course of the semester and to measure the stability and variation of rhythms among students with different mental health status. An aspect of an embodiment of the present invention then uses, among other things, the models of the rhythms to classify the mental health status of students at the end of the semester. The second study uses physio-behavioral data from 11 volunteers who wore an OURA smart ring for 30 to 323 days. We test the framework's ability to detect long-term cycles in participants' biobehavioral data and to extract commonality and differences in those cycles. We then use each person's significant cyclic periods in modeling individual rhythms and further predicting average daily readiness. Our research makes, but is not limited thereto, the following contributions:

(1) We introduce the first computational framework for modeling biobehavioral rhythms to the mobile and ubiquitous computing community that provides the ability to:
(a) flexibly process massive sensor data in different time granularity thus providing the ability to model and observe short- and long-term rhythmic behavior.
(b) identify variation and stability in individual and groups of time series data.
(c) help observe the impact of cyclic biobehavioral parameters in revealing and predicting different outcomes (e.g., health).

(2) We demonstrate the framework's ability to generate new knowledge and findings via rigorous micro- and macro-level modeling of human rhythms from mobile and wearable data streams collected in the wild and using them to assess and predict different life and health outcomes. In particular, we are the first to explore and model biobehavioral rhythms in college students and to highlight differences in rhythms among students with different mental health status. We are also the first to explore discovering of long-term personal cycles in individuals' biobehavioral data collected from consumer devices in the wild.

In the following sections, we describe related work in the domain of mobile health and behavior modeling and discuss the motivation for modeling cyclic human behavior and its potential role in revealing health status. We then present our computational framework followed by case studies in modeling biobehavioral rhythms and exploring the role of those models in predicting mental health and readiness. We discuss the feasibility and flexibility of the framework in incorporating different analytic approaches and providing insights for building rhythm-aware technology.

An aspect of an embodiment of the present invention provides, among other things, a computer-implemented method for modeling biobehavioral rhythms of a subject. The method may comprise: receiving sensor data collected from a mobile device and/or wearable device; extracting specified sensor features from the received sensor data; modeling biobehavioral rhythms for each of the extracted specified sensor features to provide modeled biobehavioral rhythm data of the subject; determining rhythmicity characteristics of cyclical behavior of the modeled biobehavioral rhythm data of the subject; measuring stability of the determined rhythmicity characteristics of the subject across different time windows and/or across different populations to determine the deviation of the subject's rhythmicity characteristics from normal rhythmicity characteristics to predict health status and/or readiness status of the subject using a machine learning module; and transmitting the predication of health status and/or readiness status to a secondary source.

An aspect of an embodiment of the present invention provides, among other things, a system configured for modeling biobehavioral rhythms of a subject. The system may comprise: a computer processor; and a memory configured to store instructions that are executable by the computer processor, wherein the processor is configured to execute the instructions to: receive sensor data collected from a mobile device and/or wearable device; extract specified sensor features from the received sensor data; model biobehavioral rhythms for each of the extracted specified sensor features to provide modeled biobehavioral rhythm data of the subject; determine rhythmicity characteristics of cyclical behavior of the modeled biobehavioral rhythm data of the subject; measure stability of the determined rhythmicity characteristics of the subject across different time windows and/or across different populations to determine the deviation of the subject's rhythmicity characteristics from normal rhythmicity characteristics to predict health status and/or readiness status of the subject using a machine learning module; and transmit the predication of health status and/or readiness status to a secondary source.

An aspect of an embodiment of the present invention provides, among other things, a computer program product, comprising a non-transitory computer-readable storage medium containing computer-executable instructions for modeling biobehavioral rhythms of a subject. The instructions causing the computer to: receive sensor data collected from a mobile device and/or wearable device; extract specified sensor features from the received sensor data; model biobehavioral rhythms for each of the extracted specified sensor features to provide modeled biobehavioral rhythm data of the subject; determine rhythmicity characteristics of cyclical behavior of the modeled biobehavioral rhythm data of the subject; measure stability of the determined rhythmicity characteristics of the subject across different time windows and/or across different populations to determine the deviation of the subject's rhythmicity characteristics from normal rhythmicity characteristics to predict health status and/or readiness status of the subject using a machine learning module; and transmit the predication of health status and/or readiness status to a secondary source.

An aspect of an embodiment of the present invention provides, among other things, a technique for providing biobehavioral rhythm models that generate a series of characteristic features which are further used for measuring stability in biobehavioral rhythms and to predict different outcomes such as health status through a machine learning component. A computational framework is provided for modeling biobehavioral rhythms from mobile and wearable data streams that rigorously processes sensor streams, detects periodicity in data, models rhythms from that data and uses the cyclic model parameters to predict an outcome. The framework can reliably discover various periods of different length in data, extract cyclic biobehavioral characteristics through exhaustive modeling of rhythms for each sensor feature; and provide the ability to use different combination of sensors and data features to predict an outcome.

The invention itself, together with further objects and attendant advantages, will best be understood by reference to the following detailed description, taken in conjunction with the accompanying drawings.

These and other objects, along with advantages and features of various aspects of embodiments of the invention disclosed herein, will be made more apparent from the description, drawings and claims that follow.

### BRIEF DESCRIPTION OF THE DRAWINGS

The foregoing and other objects, features and advantages of the present invention, as well as the invention itself, will be more fully understood from the following description of preferred embodiments, when read together with the accompanying drawings.

The accompanying drawings, which are incorporated into and form a part of the instant specification, illustrate several aspects and embodiments of the present invention and, together with the description herein, serve to explain the principles of the invention. The drawings are provided only for the purpose of illustrating select embodiments of the invention and are not to be construed as limiting the invention.

FIG. 1 schematically illustrates a system or method for the computational framework for modeling rhythms from mobile and wearable data streams and using the rhythm parameters for prediction of an outcome (e.g., health).

FIG. 2 schematically illustrates the segmentation of time series with time windows (tw) and time chunks (tc).

FIG. 3 graphically represents the rhythm parameters that can be extracted from the model generated by the periodic function.

FIG. 4 graphically illustrates the correlogram and correlation coefficients (r).

FIG. 5 schematically illustrates a system or method for measuring rhythm stability parameters.

FIG. 6 schematically illustrates the size of a time window is 2 weeks, which segments the semester into roughly 8 time windows.

FIGS. 7A and 7B graphically illustrates correlograms of feature num_restless_bout (number of restless periods in sleep) in time window 4 for two students (FIG. 7(A): a student in L_Pre1_Post1, FIG. 7(B): a student in L_Pre1_Post2).

FIGS. 8A and 8B graphically illustrates the plots that show the percentage of participants with 24-hour as the dominant rhythm (y-axis) in each mental health group (FIG. 8(A): loneliness, FIG. 8(B): depression) for each time chunk of length 3 (x-axis). The data point at x=i corresponds to the time chunk of length 3 starting at tw (i.e., tc₃ᵢ). It represents the percentage of participants with 24-hour as the dominant rhythm in all the 3 time windows twᵢ, twᵢ₊₁, twᵢ₊₂.

FIGS. 9A and 9B graphically illustrates the heatmap displays the largest F1 score in the loneliness (FIG. 9(A)) prediction model and depression (FIG. 9(B)) prediction model trained by a combination of different single sensor features and time windows.

FIG. 10(A) graphically illustrates the heatmap that displays the largest F1 score in the loneliness prediction model trained by a combination of different multiple sensor features and time windows and FIG. 10(B) graphically illustrates the heatmap that displays the best model which is obtained from Logistic Regression using the rhythm parameters.

FIG. 11 graphically illustrates the 1 to 11 boxplots display the minimum, median, maximum, and quartile of the daily readiness scores for each participant. Most daily readiness scores are clustered in the range from 70 to 85.

FIG. 12 graphically illustrates the histograms from 1 to 11 display the distribution of the daily readiness scores for each participant, and the last bar plot shows the duration of each participant's data collection.

FIG. 13 is a block diagram illustrating an example of a machine upon which one or more aspects of embodiments of the present invention can be implemented.

FIG. 14 is a flow diagram of a method for modeling biobehavioral rhythms of a subject.

### DETAILED DESCRIPTION OF EXEMPLARY EMBODIMENTS

An aspect of an embodiment of the present invention provides a system, method and computer readable medium for, among other things, a computational framework for modeling biobehavioral rhythms from mobile and wearable data streams that rigorously processes sensor streams, detects periodicity in data, models rhythms from that data and uses the cyclic model parameters to predict an outcome. Our evaluation of the framework using two different case studies shows that, but is not limited thereto, in addition to detection of rhythmicity, the framework can reliably discover various periods of different length in data, extract cyclic biobehavioral characteristics through exhaustive modeling of rhythms for each sensor feature; and provide the ability to use different combination of sensors and data features to predict an outcome. The machine learning analyses for predicting mental health and readiness demonstrated the ability of our framework to process a massive number of data streams to build and analyze micro-rhythmic models for each sensor feature and combinations of features and highlighted dominant rhythmic features for prediction of the outcome of interest. The case studies also provided novel findings that were not observed in similar studies. These results show the feasibility of our computational modeling framework for studying different outcomes and extracting new knowledge through modeling biobehavioral rhythms.

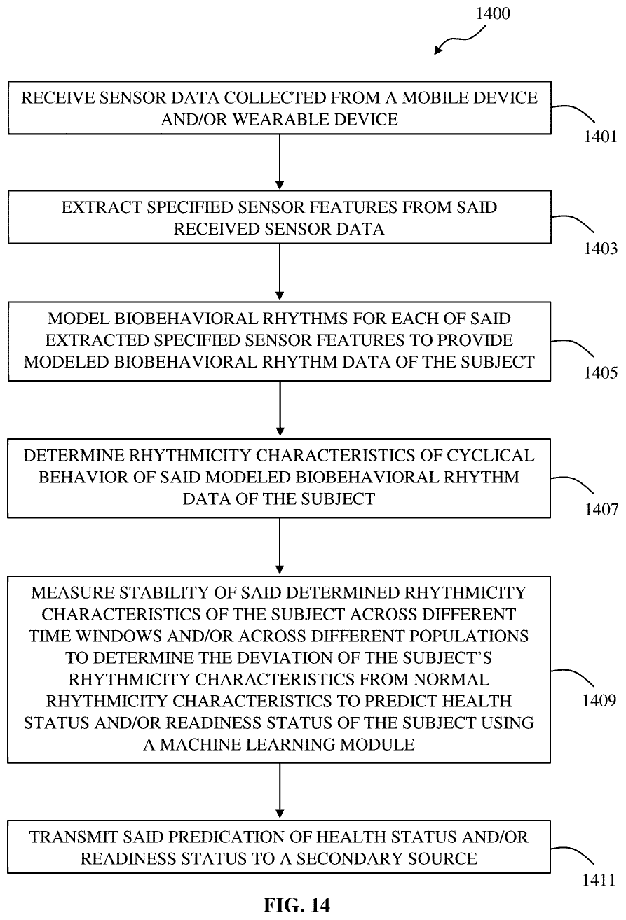

FIG. 14 is a flow diagram of a method 1400 for modeling biobehavioral rhythms of a subject. The method 1400 can be performed by a system of one or more appropriately-programmed computers in one or more locations. At step 1401, the system receives sensor data collected from a mobile device and/or wearable device. At step 1403, the system extracts specified sensor features from the received sensor data. At step 1405, the system models biobehavioral rhythms for each of the extracted specified sensor features to provide modeled biobehavioral rhythm data of the subject. At step 1407, the system determines rhythmicity characteristics of cyclical behavior of the modeled biobehavioral rhythm data of the subject. At step 1409, the system measures stability of the determined rhythmicity characteristics of the subject across different time windows and/or across different populations to determine the deviation of the subject's rhythmicity characteristics from normal rhythmicity characteristics to predict health status and/or readiness status of the subject using a machine learning module. At step 1411, the system transmits the predication of health status and/or readiness status to a secondary source.

An aspect of an embodiment of the present invention provides a system, method and computer readable medium for, among other things, a computational framework for modeling biobehavioral rhythms from mobile and wearable data streams.

An aspect of an embodiment of the present invention provides a system, method and computer readable medium for, among other things, a computational framework for modeling biobehavioral rhythms from mobile and wearable data streams that rigorously processes sensor streams, detects periodicity in data, models rhythms from that data, and uses the cyclic model parameters to predict an outcome.

An aspect of an embodiment of the present invention provides a system, method and computer readable medium for, among other things, a machine learning analysis for predicting mental health status demonstrated the framework's ability to process a massive number of data streams to build and analyze micro-rhythmic models for each sensor feature and combinations of features and highlighted dominant rhythmic features for prediction of mental health status for each sensor across time windows.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

It should be appreciated that any of the components or modules referred to with regards to any of the present invention embodiments discussed herein, may be integrally or separately formed with one another. Further, redundant functions or structures of the components or modules may be implemented. Moreover, the various components may be communicated locally and/or remotely with any user/operator/customer/client or machine/system/computer/processor. Moreover, the various components may be in communication via wireless and/or hardwire or other desirable and available communication means, systems and hardware. Moreover, various components and modules may be substituted with other modules or components that provide similar functions.

It should be appreciated that the device and related components discussed herein may take on all shapes along the entire continual geometric spectrum of manipulation of x, y and z planes to provide and meet the environmental, anatomical, and structural demands and operational requirements. Moreover, locations and alignments of the various components may vary as desired or required.

It should be appreciated that various sizes, dimensions, contours, rigidity, shapes, flexibility and materials of any of the components or portions of components in the various embodiments discussed throughout may be varied and utilized as desired or required. It should be appreciated that while some dimensions are provided on the aforementioned figures, the device may constitute various sizes, dimensions, contours, rigidity, shapes, flexibility and materials as it pertains to the components or portions of components of the device, and therefore may be varied and utilized as desired or required.

It must also be noted that, as used in the specification and the appended claims, the singular forms “a,” “an” and “the” include plural referents unless the context clearly dictates otherwise. Ranges may be expressed herein as from “about” or “approximately” one particular value and/or to “about” or “approximately” another particular value. When such a range is expressed, other exemplary embodiments include from the one particular value and/or to the other particular value.

By “comprising” or “containing” or “including” is meant that at least the named compound, element, particle, or method step is present in the composition or article or method, but does not exclude the presence of other compounds, materials, particles, or method steps, even if the other such compounds, material, particles, or method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

Some references, which may include various patents, patent applications, and publications, are cited in a reference list and discussed in the disclosure provided herein. The citation and/or discussion of such references is provided merely to clarify the description of the present disclosure and is not an admission that any such reference is “prior art” to any aspects of the present disclosure described herein. In terms of notation, “[n]” corresponds to the n^{th} reference in the list. All references cited and discussed in this specification are incorporated herein by reference in their entireties and to the same extent as if each reference was individually incorporated by reference.

It should be appreciated that as discussed herein, a subject may be a human or any animal. It should be appreciated that an animal may be a variety of any applicable type, including, but not limited thereto, mammal, veterinarian animal, livestock animal or pet type animal, etc. As an example, the animal may be a laboratory animal specifically selected to have certain characteristics similar to human (e.g. rat, dog, pig, monkey), etc. It should be appreciated that the subject may be any applicable human patient, for example.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g. 1 to 5 includes 1, 1.5, 2, 2.75, 3, 3.90, 4, 4.24, and 5). Similarly, numerical ranges recited herein by endpoints include subranges subsumed within that range (e.g. 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

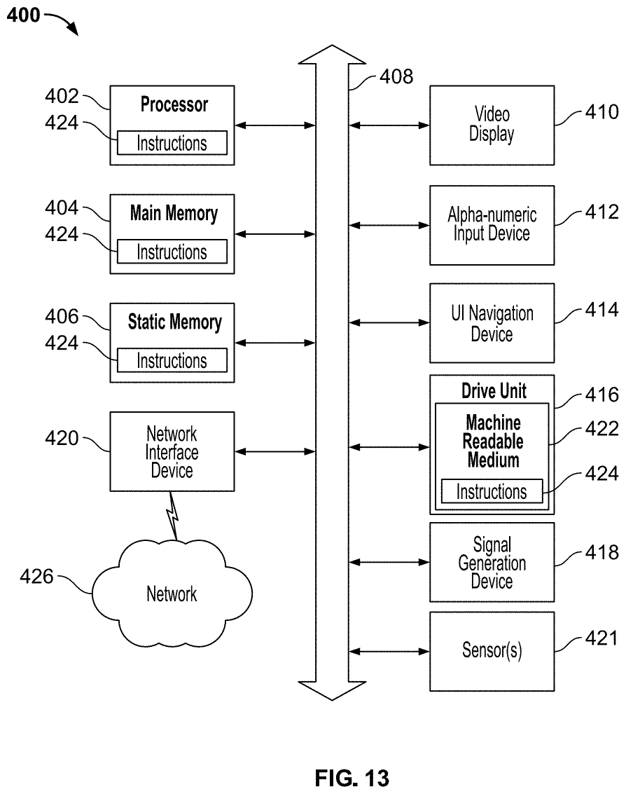

FIG. 13 is a block diagram illustrating an example of a machine upon which one or more aspects of embodiments of the present invention can be implemented. FIG. 13 illustrates a block diagram of an example of a machine 400 upon which one or more aspects of embodiments (e.g., discussed methodologies) can be implemented (e.g., run). FIG. 13 represents an aspect of an embodiment of the present invention that includes a system, method, and computer readable medium that provides, but is not limited thereto: a) a computational framework for modeling biobehavioral rhythms from mobile and wearable data streams; b) computational framework for modeling biobehavioral rhythms from mobile and wearable data streams that rigorously process sensor streams, detects periodicity in data, model rhythms from that data, and uses the cyclic model parameters to predict an outcome; and/or c) machine learning analysis for predicting mental health status that demonstrates the framework's ability to process a massive number of data streams to build and analyze micro-rhythmic models for each sensor feature and combinations of features and highlighted dominant rhythmic features for prediction of mental health status for each sensor across time windows, and which illustrates a block diagram of an example machine 400 upon which one or more embodiments (e.g., discussed methodologies) can be implemented (e.g., run).

Examples of machine 400 can include logic, one or more components, circuits (e.g., modules), or mechanisms. Circuits are tangible entities configured to perform certain operations. In an example, circuits can be arranged (e.g., internally or with respect to external entities such as other circuits) in a specified manner. In an example, one or more computer systems (e.g., a standalone, client or server computer system) or one or more hardware processors (processors) can be configured by software (e.g., instructions, an application portion, or an application) as a circuit that operates to perform certain operations as described herein. In an example, the software can reside (1) on a non-transitory machine readable medium or (2) in a transmission signal. In an example, the software, when executed by the underlying hardware of the circuit, causes the circuit to perform the certain operations.

In an example, a circuit can be implemented mechanically or electronically. For example, a circuit can comprise dedicated circuitry or logic that is specifically configured to perform one or more techniques such as discussed above, such as including a special-purpose processor, a field programmable gate array (FPGA) or an application-specific integrated circuit (ASIC). In an example, a circuit can comprise programmable logic (e.g., circuitry, as encompassed within a general-purpose processor or other programmable processor) that can be temporarily configured (e.g., by software) to perform the certain operations. It will be appreciated that the decision to implement a circuit mechanically (e.g., in dedicated and permanently configured circuitry), or in temporarily configured circuitry (e.g., configured by software) can be driven by cost and time considerations.

Accordingly, the term “circuit” is understood to encompass a tangible entity, be that an entity that is physically constructed, permanently configured (e.g., hardwired), or temporarily (e.g., transitorily) configured (e.g., programmed) to operate in a specified manner or to perform specified operations. In an example, given a plurality of temporarily configured circuits, each of the circuits need not be configured or instantiated at any one instance in time. For example, where the circuits comprise a general-purpose processor configured via software, the general-purpose processor can be configured as respective different circuits at different times. Software can accordingly configure a processor, for example, to constitute a particular circuit at one instance of time and to constitute a different circuit at a different instance of time.

In an example, circuits can provide information to, and receive information from, other circuits. In this example, the circuits can be regarded as being communicatively coupled to one or more other circuits. Where multiple of such circuits exist contemporaneously, communications can be achieved through signal transmission (e.g., over appropriate circuits and buses) that connect the circuits. In embodiments in which multiple circuits are configured or instantiated at different times, communications between such circuits can be achieved, for example, through the storage and retrieval of information in memory structures to which the multiple circuits have access. For example, one circuit can perform an operation and store the output of that operation in a memory device to which it is communicatively coupled. A further circuit can then, at a later time, access the memory device to retrieve and process the stored output. In an example, circuits can be configured to initiate or receive communications with input or output devices and can operate on a resource (e.g., a collection of information).

The various operations of method examples described herein can be performed, at least partially, by one or more processors that are temporarily configured (e.g., by software) or permanently configured to perform the relevant operations. Whether temporarily or permanently configured, such processors can constitute processor-implemented circuits that operate to perform one or more operations or functions. In an example, the circuits referred to herein can comprise processor-implemented circuits.

Similarly, the methods described herein can be at least partially processor-implemented. For example, at least some of the operations of a method can be performed by one or processors or processor-implemented circuits. The performance of certain of the operations can be distributed among the one or more processors, not only residing within a single machine, but deployed across a number of machines. In an example, the processor or processors can be located in a single location (e.g., within a home environment, an office environment or as a server farm), while in other examples the processors can be distributed across a number of locations.

The one or more processors can also operate to support performance of the relevant operations in a “cloud computing” environment or as a “software as a service” (SaaS). For example, at least some of the operations can be performed by a group of computers (as examples of machines including processors), with these operations being accessible via a network (e.g., the Internet) and via one or more appropriate interfaces (e.g., Application Program Interfaces (APIs).)

Example embodiments (e.g., apparatus, systems, or methods) can be implemented in digital electronic circuitry, in computer hardware, in firmware, in software, or in any combination thereof. Example embodiments can be implemented using a computer program product (e.g., a computer program, tangibly embodied in an information carrier or in a machine readable medium, for execution by, or to control the operation of, data processing apparatus such as a programmable processor, a computer, or multiple computers).

A computer program can be written in any form of programming language, including compiled or interpreted languages, and it can be deployed in any form, including as a stand-alone program or as a software module, subroutine, or other unit suitable for use in a computing environment. A computer program can be deployed to be executed on one computer or on multiple computers at one site or distributed across multiple sites and interconnected by a communication network.

In an example, operations can be performed by one or more programmable processors executing a computer program to perform functions by operating on input data and generating output. Examples of method operations can also be performed by, and example apparatus can be implemented as, special purpose logic circuitry (e.g., a field programmable gate array (FPGA) or an application-specific integrated circuit (ASIC)).

The computing system can include clients and servers. A client and server are generally remote from each other and generally interact through a communication network. The relationship of client and server arises by virtue of computer programs running on the respective computers and having a client-server relationship to each other. In embodiments deploying a programmable computing system, it will be appreciated that both hardware and software architectures require consideration. Specifically, it will be appreciated that the choice of whether to implement certain functionality in permanently configured hardware (e.g., an ASIC), in temporarily configured hardware (e.g., a combination of software and a programmable processor), or a combination of permanently and temporarily configured hardware can be a design choice. Below are set out hardware (e.g., machine 400) and software architectures that can be deployed in example embodiments.

In an example, the machine 400 can operate as a standalone device or the machine 400 can be connected (e.g., networked) to other machines.

In a networked deployment, the machine 400 can operate in the capacity of either a server or a client machine in server-client network environments. In an example, machine 400 can act as a peer machine in peer-to-peer (or other distributed) network environments. The machine 400 can be a personal computer (PC), a tablet PC, a set-top box (STB), a Personal Digital Assistant (PDA), a mobile telephone, a web appliance, a network router, switch or bridge, or any machine capable of executing instructions (sequential or otherwise) specifying actions to be taken (e.g., performed) by the machine 400. Further, while only a single machine 400 is illustrated, the term “machine” shall also be taken to include any collection of machines that individually or jointly execute a set (or multiple sets) of instructions to perform any one or more of the methodologies discussed herein.

Example machine (e.g., computer system) 400 can include a processor 402 (e.g., a central processing unit (CPU), a graphics processing unit (GPU) or both), a main memory 404 and a static memory 406, some or all of which can communicate with each other via a bus 408. The machine 400 can further include a display unit 410, an alphanumeric input device 412 (e.g., a keyboard), and a user interface (UI) navigation device 411 (e.g., a mouse). In an example, the display unit 810, input device 417 and UI navigation device 414 can be a touch screen display. The machine 400 can additionally include a storage device (e.g., drive unit) 416, a signal generation device 418 (e.g., a speaker), a network interface device 420, and one or more sensors 421, such as a global positioning system (GPS) sensor, compass, accelerometer, or other sensor.

The storage device 416 can include a machine readable medium 422 on which is stored one or more sets of data structures or instructions 424 (e.g., software) embodying or utilized by any one or more of the methodologies or functions described herein. The instructions 424 can also reside, completely or at least partially, within the main memory 404, within static memory 406, or within the processor 402 during execution thereof by the machine 400. In an example, one or any combination of the processor 402, the main memory 404, the static memory 406, or the storage device 416 can constitute machine readable media.

While the machine readable medium 422 is illustrated as a single medium, the term “machine readable medium” can include a single medium or multiple media (e.g., a centralized or distributed database, and/or associated caches and servers) that configured to store the one or more instructions 424. The term “machine readable medium” can also be taken to include any tangible medium that is capable of storing, encoding, or carrying instructions for execution by the machine and that cause the machine to perform any one or more of the methodologies of the present disclosure or that is capable of storing, encoding or carrying data structures utilized by or associated with such instructions. The term “machine readable medium” can accordingly be taken to include, but not be limited to, solid-state memories, and optical and magnetic media. Specific examples of machine readable media can include non-volatile memory, including, by way of example, semiconductor memory devices (e.g., Electrically Programmable Read-Only Memory (EPROM), Electrically Erasable Programmable Read-Only Memory (EEPROM)) and flash memory devices; magnetic disks such as internal hard disks and removable disks; magneto-optical disks; and CD-ROM and DVD-ROM disks.

The instructions 424 can further be transmitted or received over a communications network 426 using a transmission medium via the network interface device 420 utilizing any one of a number of transfer protocols (e.g., frame relay, IP, TCP, UDP, HTTP, etc.). Example communication networks can include a local area network (LAN), a wide area network (WAN), a packet data network (e.g., the Internet), mobile telephone networks (e.g., cellular networks), Plain Old Telephone (POTS) networks, and wireless data networks (e.g., IEEE 802.11 standards family known as Wi-Fi®, IEEE 802.16 standards family known as WiMax®), peer-to-peer (P2P) networks, among others. The term “transmission medium” shall be taken to include any intangible medium that is capable of storing, encoding or carrying instructions for execution by the machine, and includes digital or analog communications signals or other intangible medium to facilitate communication of such software.

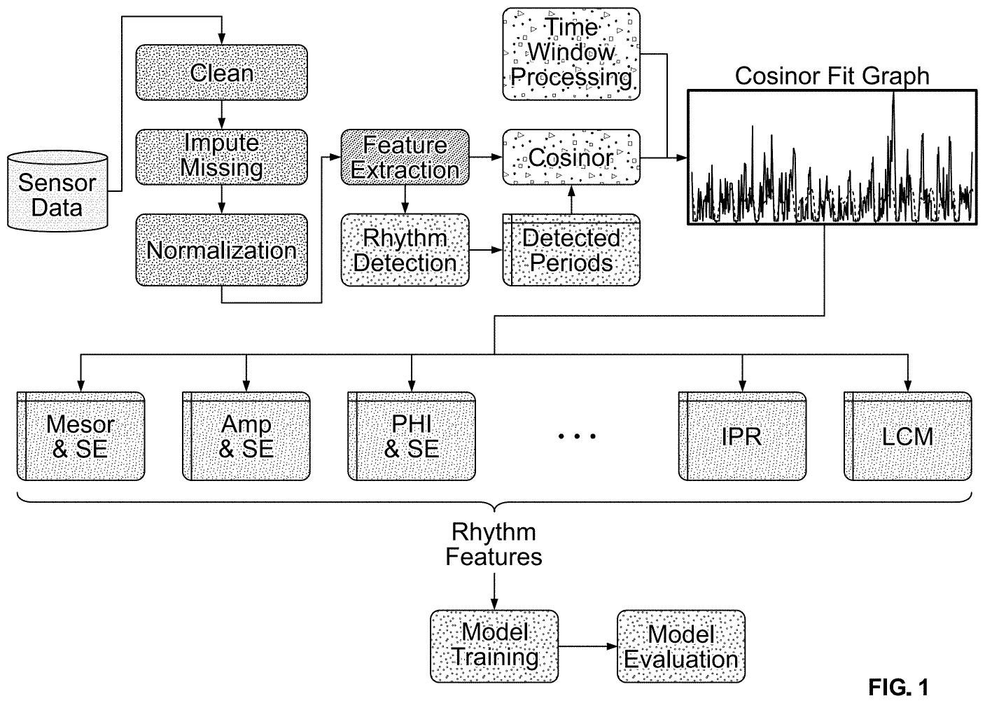

Computational Framework for Modeling Biobehavioral Rhythms. An aspect of an embodiment of the present invention provides, among other things, a framework (FIG. 1) that incorporates data streams from mobile and wearable devices including behavioral signals such as movement, audio, bluetooth, wifi, and GPS and logs of phone usage and communication (calls and messages); and biosignals such as heart rate, skin temperature, and galvanic skin response. These signals are processed and granular features that characterize biobehavioral patterns such as activity, sleep, social communication, work, and movements are extracted. The data streams of biobehavioral sensor features are segmented into different time windows of interest and sent to a rhythm discovery component that applies periodic functions on each windowed stream of the sensor feature to detect their periodicity. The detected periods are then used to model the rhythmic function that represents the time series data stream for that sensor feature. The parameters generated by the rhythmic function are used in two ways. First, they are aggregated and further processed to characterize the stability or variation in rhythms over a certain time segment. Second, they are used as features in a machine learning pipeline to predict an outcome of interest (e.g., health status). FIG. 1 schematically illustrates a system or method for the computational framework for modeling rhythms from mobile and wearable data streams and using the rhythm parameters for prediction of an outcome (e.g., health). The following sections provide details on the methods used in different components of the framework.

Time Series Segmentation. Windowing is one of the most frequently used processing methods for streams of data. A time series of length L is split into N segments based on certain criteria such as time. Our framework allows different ways to segment the time series, including the widely used tumbling windows, which are a series of fixed-sized, non-overlapping and contiguous time intervals. We call each segment a time window (tw) which is a time series of length l, where l=LIN.

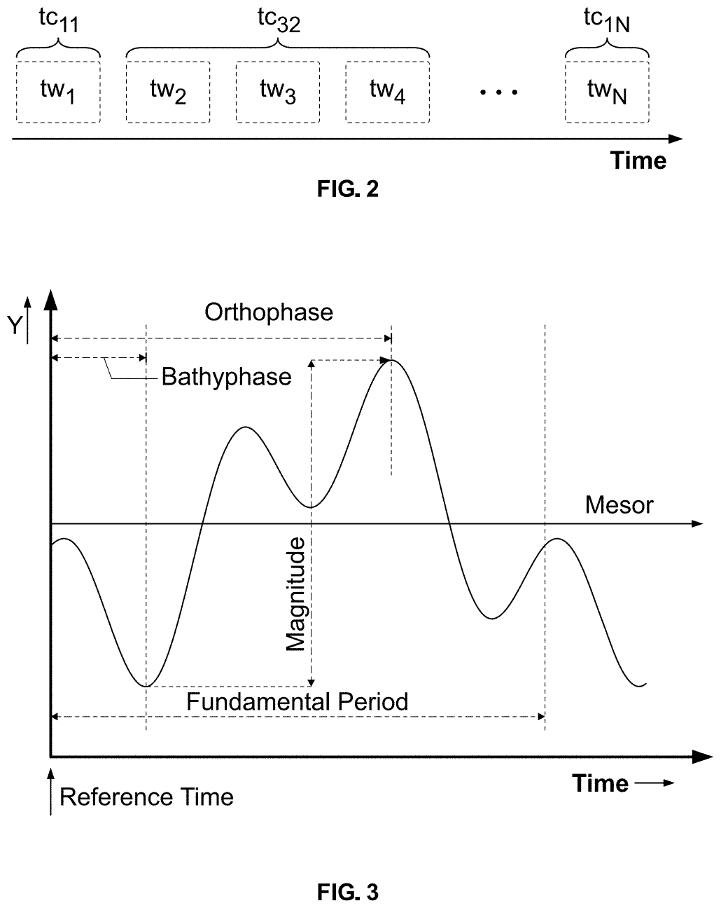

We also add a second segmentation layer to the time series where at each round k and starting point s (s=1 . . . N), we allow to combine a sequence of k consecutive time windows (k=1 . . . N) starting from time window s (twₛ) to generate time series of length k. We call these segments time chunks (tc). For example, in round k=1, the tc₁₁ is a time chunk of length one and starting point of tw₁ and tc₁₂ is a time chunk of length one and starting point tw₂ whereas for k=3, the tc₃₂ is a time chunk of length three and starting point of tw₂. Time chunks allow flexible modeling of rhythms in different time periods over the length of the time series. FIG. 2 illustrates the time segmentation process. FIG. 2 schematically illustrates the segmentation of time series with time windows (tw) and time chunks (tc).

Detection of Rhythmicity. One of the first steps in modeling biobehavioral rhythms is identifying rhythmicity in time series data. We use two main methods for detecting and observing cyclic behavior, namely Autocorrelation and Periodogram.

Autocorrelation. Autocorrelation is a reliable analytical method for recognizing periodicities [20]. It calculates the correlation coefficient between a time series and its lagged version to measure the similarity between them over consecutive time intervals. Formally, the autocorrelation function (ACF) between two values yₜ, yₜ₋ₖ in a time series yₜ is defined as

Corr(yₜ,yₜ₋ₖ),k=1,2, . . . , (1)

where k is the time gap and is called the lag [45]. In each iteration, the two time series are shifted by k points until one third of data is parsed. If the time series is rhythmic, the coefficient values increase and decrease in regular intervals and significant correlations indicate strong periodicity in data. The autocorrelation sequence of a periodic signal has the same cyclic characteristics as the signal itself. Thus, autocorrelation can help verify the presence of cycles and determine the periods. It has been empirically applied on various types of time series data from different fields and was shown to be dependable and exact in the tested situations [47, 55].

Periodogram. One of the key steps in the rhythm discovery process is estimation of the length of period for each rhythm. Many different techniques and algorithms for determining the period of a cycle have been developed including the Fourier-transform based methods such as Fast Fourier Transform [6], Non-Linear Least Squares [58] and Spectrum Resampling [13]. Other frequently used methods are Enright and Lomb-Scargle periodograms [23, 39], mFourfit [22], Maximum Entropy Spectral Analysis [11], and Chi-Square periodograms [57]. All of these methods come with different assumptions and with different levels of complexity [51]. For example, Spectrum Sampling has outperformed the usual Fourier approximation methods and has shown more robustness towards non-sinusoidal and noisy cycles [64]. It has also been used to detect changes in period length, which allows for estimation of variance in different periods, as frequently observed in practice. These functionalities, however, have made the algorithm slow and computationally expensive [64].

Arthur Schuster used Fourier analysis to evaluate periodicity in meteorological phenomena and introduced the term ‘periodogram’ [56]. The method was first applied to the study of circadian rhythms in the early 1950s to quantify free-running rhythms of mice after blinding [34]. Periodograms provide a measure of strength and regularity of the underlying rhythm through estimation of the spectral density of a signal. For a time series yₜ, t=1, 2, . . . , T, the spectral energy Pₖ of frequency k can be calculated as [50]:

$${P}_{k} = {( \frac{2}{T} {\sum }_{i = 1}^{T} {y}_{t} cos ( \frac{2 π kt}{T} ) )}^{2} + {( \frac{2}{T} {\sum }_{i = 1}^{T} {y}_{t} sin ( \frac{2 π kt}{T} ) )}^{2} . \qquad ( 2 )$$

The periodogram uses a Fourier Transform to convert a signal from the time domain to the frequency domain. A Fourier analysis is a method for expressing a function as a sum of periodic components, and for recovering the time series from those components. The dominant frequency corresponds to the periodicity in the pattern.

Modeling Rhythms. The next step in our framework is modeling the rhythmic behavior of a time series data which is done via a periodic function. Each periodic function is among others specified by its period, average level (MESOR), oscillation degree (Amplitude), and time of oscillation optimal (Phase) [33]. The following rhythm parameters can be extracted from the model generated by the periodic function (as graphically illustrated in FIG. 3) [12, 24, 38]:
Fundamental period: Periodic sequences are usually made up of multiple periodic components. The fundamental period measures the time during an overall cycle.
MESOR is the midline of the oscillatory function. When the sampling interval is equal, the MESOR is equal to the mean value of all cyclic data points.
Amplitude (Amp) refers to the maximum value a single periodic component can reach. The amplitude of a symmetrical wave is half of its range of up and down oscillation.
Magnitude refers to the difference between the maximum value and the minimum value within a fundamental period. If a periodic sequence only contains one periodic component, amplitude equals half of the magnitude.
Acrophase (PHI) refers to the time distance between the defined reference time point and the first time point in a cycle where the peak occurs with a period of a single periodic component.
Orthophase refers to the time distance between the defined reference time point and the first time point in a cycle where the peak occurs with a fundamental period. When the time sequence only contains one periodic component, orthophase equals to acrophase.
Bathyphase refers to the time distance between the defined reference time point and the first time point in a cycle where the trough occurs with a fundamental period.
P-value (P) indicates the overall significance of the model fitted by a single period and comes from the F-test comparing the built model with the zero-amplitude model.
Percent rhythm (PR) is the equivalent to the coefficient of determination (denoted by R²) representing the proportion of overall variance accounted for by the fitted model.
Integrated p-value (IP) represents the significance of the model fitted by the entire periods.
Integrated percent rhythm (IPR) is the R² of the model fitted by the entire periods.
The longest cycle of the model (LCM) equals to the least common multiple of all single periods.

The most fundamental method for modeling rhythms with known periods is Cosinor, a periodic regression function first developed by Halberg et al [31] that uses the least squares method to fit one or several cosine curves with or without polynomial terms to a single time series. It uses the following cosine function to model the time series [24]:

$${y}_{i} = M + {\sum }_{c = 1}^{C} {A}_{c} cos ( {ω}_{c} {t}_{i} + {ϕ}_{c} ) + {e}_{i} . \qquad ( 3 )$$

where yᵢ is the observed value at time tᵢ; M presents the MESOR; tᵢ is the sampling time; C is the set of all periodic components; A_{c}, ω_{c}, ϕ_{c} respectively presents the amplitude, frequency, and acrophase of each periodic components; and eᵢ is the error term. In addition to the parameters described above, Cosinor outputs the standard error (SE) for MESOR, amplitude, and acrophase respectively.

The Cosinor models can be generated for one time series (single Cosinor—individual model) or for a group of time series (population-mean Cosinor—population model) through aggregation of rhythm parameters obtained from single Cosinors. Cosinor models have been used to characterize circadian rhythms and to compute relevant parameters with their confidence limits. The model outputs the significance of the period and it is proved that if P≤0.05, the assumed period actually exists. Our Co framework allows for different periodic functions to be applied to the time series data using the detected periods from the previous step. We then use the rhythmic parameters measured by the Cosinor model in our machine learning pipeline as described in the next section.

Measuring Rhythm Stability. An important aspect of biobehavioral rhythms is their stability or deviations from normal. As mentioned previously, disruption in biological rhythms is associated with different health outcomes. As such, in our framework, we develop methods to measure the stability and variations in rhythms among individual people over different time periods (within-person) and across different population groups (between-person). Our methods employ models built by Autocorrelation and Cosinor functions to discover and measure the stability of the time series over its length. One of the goals behind developing two different methods is to compare their performance in measuring rhythm stability. Besides, each method may provide unique insights that cannot be drawn from the other method.

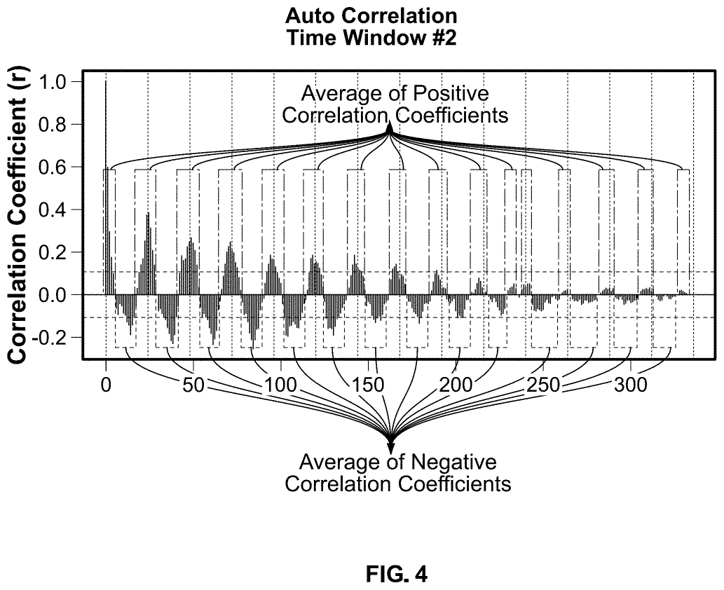

Autocorrelation Sequence Stability Score (CORRES). Recall that Autocorrelation iteratively calculates the correlation coefficient between the time series and its lagged version from start to end. The coefficient values (r) create a sequence that can be plotted by a correlogram (FIG. 4) that provides a visual representation of the rhythmicity in data. FIG. 4 graphically illustrates the correlogram and correlation coefficients (r). The peaks above the horizontal-dashed line in FIG. 4 indicate significant correlations, and rapid decay in the amplitude of peaks indicates variation in data. To measure fluctuations in rhythms, we develop a new method to extract variability attributes from the autocorrelation sequence and further measure an overall stability score for the time series being analyzed. The process of extracting the variability parameters from the generated autocorrelation model is as follows:
1. We process the generated autocorrelation sequence to calculate the mean and standard deviation of positive correlation coefficients and the mean and standard deviation of negative correlation coefficients generated by the Autocorrelation analysis as shown in FIG. 4.
2. We measure the length of each correlation bout, i.e., the series of consecutive positive or negative correlations (the cones in the correlogram in FIG. 4). We then calculate the min, max, mean, and standard deviation of lengths of those positive and negative bouts.
3. We identify the longest positive and negative correlation bouts in the time series and calculate the min, max, mean, and standard deviation of correlation coefficients in each of these two bouts.
4. We sort the positive correlation bouts by the highest correlation value in the bout. We then take the three positive correlation bouts with the highest correlation values and calculate the min, max, mean and standard deviation of correlation coefficients in each of the three bouts. We also calculate the same values for negative correlation bouts.

To calculate the stability, we first compute the ratio of aforementioned attributes: the positive correlation attribute over the negative correlation for that attribute (e.g., the average length of positive correlation bouts over the average length of negative correlation bouts) which provide the local stability score for that attribute. We then calculate the overall stability score by aggregating the scores of all those attributes. More formally, let aᵢ be the attribute in the above list (e.g., average correlation) and pos(aᵢ) and neg(aᵢ) be the corresponding positive and negative values of aᵢ. The local stability score (loc_{css}) is calculated as:

$${loc}_{css} = \vert \frac{pos ( {a}_{i} )}{neg ( {a}_{i} )} \vert .$$

The total stability score for a time window t is

tw_{css}=Σᵢ₌₁^{I}loc_{css}(aᵢ)

where I is the number of attributes. The Horizontal CORRES (HORRES) is then measured for individual time series ts of K consecutive time windows, where

$${ts}_{css} = \frac{{\sum }_{k = 1}^{K} {tw}_{css}}{K} .$$

Vertical CORRES (VORRES) is measured for groups of time series (different population groups) where the CORRES score for each population group g of size N_{g} in each time window tw is measured as

$${g}_{css} = \frac{{\sum }_{g = 1}^{G} {tw}_{css}}{{\sum }_{g = 1}^{G} {N}_{g}}$$

where G is the total number of population groups.

Variance of Rhythmic Parameters (COSANOVA). As mentioned previously, Cosinor analysis can be applied to a single or group of time series. The latter is called population-mean Cosinor. We develop a second method which we call COSANOVA for measuring rhythms stability among different populations through measuring the variance of rhythm parameters obtained from the population cosinor model. COSANOVA measures the variance of MESOR, amplitude, and acrophase for each population across consecutive time windows—Horizontal COSANOVA (HANOVA), and for each time window across different populations—Vertical COSANOVA (VANOVA). The COSANOVA stability score is calculated using the average p values from HANOVA and VANOVA. In HANOVA, the mean value and the standard error of the rhythm parameters in K consecutive time windows (twt−twₜ₊ₖ) are used to calculate the significance (p value) of the variance between the means. In VANOVA, on the other hand, the mean and standard error of the rhythm parameters among G population groups are compared for the significance of variance in each time window/wₜ. In other words, HANOVA defines group level stability, and VANOVA defines time window level stability. If HANOVA score is greater than the significance level (e.g., 0.05), the group rhythm is stable. Similarly, if VANOVA score is greater than the significance level (e.g., 0.05), the time window rhythm is stable. The scores greater than the significance level mean the variance of rhythm is not significant.

Let pᵣ be the significance of variance for each rhythm parameter across K time windows for population g. The HANOVA score for this population is calculated as

$${g}_{hanova} = \frac{{\sum }_{r = 1}^{R} {p}_{r}}{R}$$

where R is the number of rhythm parameters. The VANOVA score for time window twₖ is measured as

$${tw}_{vanova} = \frac{{\sum }_{r = 1}^{R} {\sum }_{g = 1}^{G} {p}_{r}}{G . R}$$

where G is the number of population groups. The COSANOVA score for each sensor feature f is the

$${f}_{cosanova} = \frac{{\sum }_{k = 1}^{K} {tw}_{vanova} {\sum }_{g = 1}^{G} {g}_{hanova}}{G . K} .$$

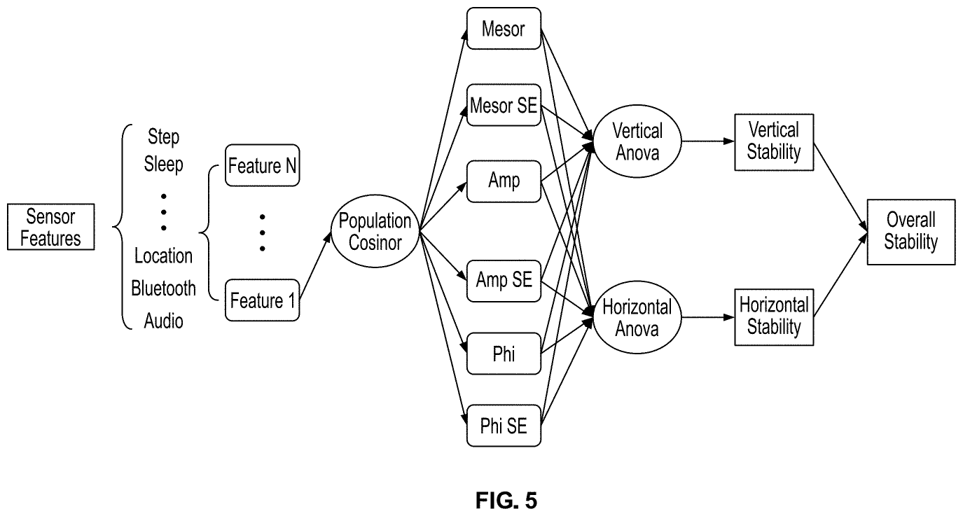

We then calculate the percentage of sensor features with stable rhythms in each population group and across time windows, which provides an overall stability score for the entire population (i.e., all groups together). FIG. 5 illustrates the pipeline of calculating the stability score for the rhythms of each sensor feature. For instance, FIG. 5 schematically illustrates a system or method for the method or system for measuring rhythm stability parameters.

Machine Learning Method. The machine learning component of the framework uses the parameters obtained from modeling the rhythm of each sensor feature to generate datasets for training and testing of an outcome of interest, e.g., health. The pipeline processes and handles missing values both in sensor and rhythm features across different time windows, selects important rhythm features as part of the training process and builds machine learning models for prediction of the outcome. The following sections describe the details of each step.

Handling Missing Values. Given the streams of data from multiple sources, the framework handles missing data for each sensor stream and each time window. We remove any sensor feature if the percent of its missing data is greater than a threshold (e.g., 30%). For remaining sensor features, we perform nearest-neighbor linear interpolation [8] to fill in missing values. For example, if there are 3 missing data points between 10 and 50, then the 3 missing points are filled with 20, 30 and 40 respectively. Given that the first and last data points cannot be imputed using this method, we remove the sensor feature if the first or the last data point in the time window is missing.

We apply the same process for handling missing rhythmic features in consecutive time windows. For each rhythmic feature, we fill the value of the missing time window with nearest-neighbor linear interpolation. Let vᵢ be the value of feature in time window twᵢ. If vᵢ and v₅, the values of features in time windows tw₁ and tw₅ are present and v₂, v₃, and v₄, the feature values of tw₂, tw₃ and two are missing, then

$$diff = \frac{{υ}_{5} - {υ}_{1}}{5 - 1} , and {υ}_{2} = {υ}_{1} + diff , {υ}_{3} = {υ}_{1} + diff * 2 , and {υ}_{4} = {υ}_{1} + diff * 3.$$

For each missing time window, if none of the time windows before it has value, or none of the time windows after it has value, then this time window is not filled. After imputation, we remove any rhythmic feature with missing values more than a threshold (e.g., 30%). Algorithm 1 describes the process in more details.

| Algorithm 1: Missing value imputation | | | |
|---|---|---|---|
| Data: Input dataset D | | | |
| Find the indexes list of the existing values In | | | |
| Missing value counter: c = In[0] | | | |
| for i = 1 to len(In) do | | | |
| \| | index_diff = In[i] − In[i − 1] | | |
| \| | if index_diff > 1 then | | |
| \| | \| | value_diff = D[In[i]] − D [In[i − 1]] | |
| \| | \| | c = c + index_diff | |
| \| | \| | for In[i-1] < i < In[i] do | |
| \| | \| | \| | $D [ j ] = \frac{value\_diff}{index\_diff} \cdot ( j - In [ i ] )$ |

| | | |
|---|---|---|
| \| | \| | end |

| \| | end |
|---|---|
| end | |
| Missing rate threshold = θ | |
| Number of data points in D = N | |
| $if \frac{c}{N} > θ then$ | |
| \| | Delete D |
| else | |
| \| | return the imputed dataset |

end

Feature Selection. As mentioned in previous sections, for each type of sensor feature, a single period or a multifrequency Cosinor model is generated which outputs a list of rhythm parameters. These parameters are entered the training process for building machine learning models.

Let M be the number of sensors (s₁ . . . sₘ), FNᵢ be the number of features for sensor i and RNⱼ the corresponding number of rhythmic features for feature j in sensor i. The resulting feature space will be of M*FN*RN which is high dimensional compared to the relatively few data samples for training. As such, a reduction in the number of features is prevalent. The framework allows for integration of different feature selection methods such as Lasso, Randomized Logistic Regression (RLR), and Information Gain (IG) in the machine learning component.

Lasso is a linear regression model penalized with the L1 norm to fit the coefficients [10]. The Lasso regression prefers solutions with fewer non-zero coefficients and effectively reduces the number of features that are independent of the target variable. Through cross-validation, the lasso regression can output the importance level for each feature in the training dataset. We use a threshold value of 1e-5 to select features with Lasso, which is the default threshold in Sklearn library. Features with importance greater or equal to the threshold are kept and the rest are discarded.

Randomized Logistic Regression is developed for stability selection of features. The basic idea behind stability selection is to use a base feature selection algorithm like logistic regression to find out which features are important in bootstrap samples of the original dataset [42]. The results on each bootstrap sample are then aggregated to compute a stability score for each feature in the data. Features with a higher stability score than a threshold are selected. We use 0.25, the default threshold value in Sklearn library.

Information Gain (also referred to as Mutual Information in feature selection) measures the dependence between the features and the dependent variable (predicted outcome) [35]. Mutual information is always larger than or equal to zero, where the larger the value, the greater the relationship between the two variables. If the calculated result is zero, then the variables are independent. We set our algorithm to select 10 (the default value in Sklearn library) features with highest information gain.

Model Building and Validation. The step for building machine learning models using rhythm features of k consecutive time windows and for a population of D data samples is flexible in the framework and can incorporate different supervised and unsupervised machine learning methods such as regression, classification, and clustering. In the current version of the framework, we implement three classification methods including Logistic Regression (LR), Random Forest (RF), and Gradient Boosting (GB). The choice of algorithms is simply based on our empirical evidence of their performance on this type of data. Logistic regression [43] uses the logistic function to build a classifier. Random forest and Gradient Boosting are two branches of ensemble learning [15] which use the idea of bagging and boosting [9] respectively. Their common feature is to use the decision tree as the basic classifier and to get a robust model by combining multiple weak models. Bagging is short for boost strapped aggregation. Boost strapping is a repeated sampling method with replacement and random sampling [26]. In boosting, the training set of each iteration is unchanged but the weight of samples is changed. At each iteration, the training samples with high error rates are given higher weights, so they get more attention in the next round training.

To better understand the role of each sensor in prediction, we build models with features from single sensors alone and features from multiple sensors. We use a baseline of the majority class to measure the performance of the classifiers in prediction of the outcome. Again, the flexibility of the framework allows for incorporation of different baseline measures. Both feature selection process and building machine learning models are done in a cross-validation setting, e.g., leave one sample out [63]. The machine learning component can measure basic performance measures of accuracy, precision, recall, F1, and MCC scores to evaluate the algorithms performance. From those measures, we choose the results above baseline for each combination of feature selection and learning algorithm to further explore the prediction outcomes and to gain insights.

Evaluation. To demonstrate the capability of our framework in building rhythm models from micro- and macro-level sensor features and utilizing them in prediction tasks, we present two different cases. The first case, utilizes data from smartphone and Fitbit to explore the relationship between biobehavioral rhythms and mental health status. The second case, investigates long-term biobehavioral rhythms of data from OURA smart ring and their ability to predict readiness. We choose different analysis approaches to showcase the flexibility of the framework in handling different types of data and measuring various outcomes.

Case 1: Classification of Mental Health via Rhythm Models Using Data from Smartphone and Fitbit. We utilized a dataset of smartphone, Fitbit, and survey data collected from 138 first-year undergraduate students at an American university who were recruited for a health and well-being research study. The dataset was previously used in [19] to detect loneliness among college students. Smartphone data was collected through the AWARE framework [25] and included calls, messages, screen usage, Bluetooth, Wi-Fi, audio, and location. A Fitbit Flex2 wearable fitness tracker tracked steps, distances, calories burned, and sleep; and survey questions gathered information about physical and mental health including loneliness and depression. The survey data was collected at the beginning and at the end of the semester.

In an embodiment, our analysis was performed in two steps: First, we explored the potential of modeling and detecting rhythmicity in passively collected data from students' mobile and wearable data streams. Then we used the built rhythm models to extract features that were fed into machine learning models to explore the relationship between students' biobehavioral rhythms and their mental health. We aimed to answer the following questions:
(1) Can we observe rhythmicity in students' biobehavioral data over the course of the semester? If so, are those rhythms consistent throughout the semester or do they change during different periods?
(2) Do we observe any difference in biobehavioral rhythms among students with different health status? If so, do healthy students have more stable rhythms?
(3) How accurately can models of biobehavioral rhythms predict mental health status?
(4) What are the most important characteristics and rhythmic features that reveal change in health status?

Note that our framework provides the ability to generate a large number of observations on the micro-(sensor feature) and macro-level (sensor), but in this embodiment, we only focus on observations related to our analysis questions.

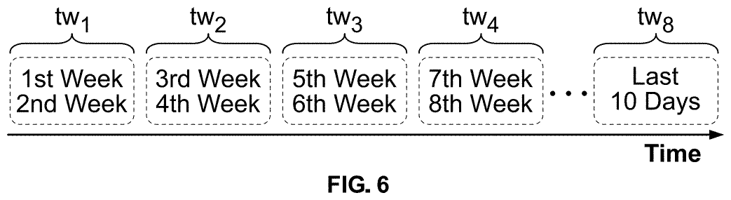

Sensor Data Processing. The dataset collected from smartphones and Fitbits consisted of time series data from multiple sensors including Bluetooth, calls, SMS, Wi-Fi, location, phone usage, steps, and sleep. We grouped this time series data into hourly bins and processed it following the approach in [17] to extract features related to mobility and activity patterns, communication and social interaction, and sleep. Examples of such features include travel distance, sleep efficiency, and movement intensity. We then split the semester data into tumbling cyclic time windows of 14 days or two weeks based on empirical evaluation of different lengths of time windows. The university semester in the studied population was roughly 16 weeks long which could by divided into 8 time windows of two weeks except the last time window that contained only 10 days of data (FIG. 6). We built a model of rhythm for each student and for each time window. FIG. 6 schematically illustrates the size of a time window is 2 weeks which segments the semester into roughly 8 time windows.

We handled missing sensor data on a per-participant per-time window basis. For each participant and each time window, we removed sensor features with more than 30% missing data. For remaining sensor features, we performed nearest-neighbor linear interpolation as described previously to fill in missing values.

Ground Truth Measures for Loneliness and Depression. In our evaluation, we focused on two mental health outcomes namely depression and loneliness. These two measures were chosen because of their longitudinal aspect, i.e., lasting for at least a few weeks to enable the investigation of 1) how biobehavioral rhythms of students with mental health conditions would differ from other students and 2) how accurately the state of those mental health conditions could be predicted from extracted rhythms.

Loneliness data was collected using the UCLA Loneliness Scale, a well-validated and commonly used measure of general feelings of loneliness [53]. The questionnaire contains 20 questions about feeling lonely and isolated using a scale of 1 (never) to 4 (always). The total loneliness scores range from 20 to 80 with higher scores indicating higher levels of loneliness. As there is no standard cutoff for loneliness scores in the literature, we followed the same approach in [19] to divide the UCLA scores into two categories where the scores of 40 and below were categorized as ‘low loneliness’ and the scores above 40 were categorized as ‘high loneliness’.

Depression was assessed using the Beck Depression Inventory-II (BDI-II) [4, 21], a widely used psychometric test for measuring the severity of depressive symptoms that has been validated for college students [21]. The BDI-II contains 21 questions, with each answer being scored on a scale of 0-3 where higher scores indicate more severe depressive symptoms. For college students, the cut-offs on this scale are 0-13 (no or minimal depression), 14-19 (mild depression), 20-28 (moderate depression) and 29-63 (severe depression) [21]. For simplicity and to be consistent with the loneliness categorization, we divided these scores into two categories where the BDI-II scores <14 were labeled as ‘not having depression’ and all BDI-II scores>=14 were labeled as ‘having depression’.

These loneliness and depression categories were used as ground truth labels in our machine learning pipeline to classify students' depression and loneliness levels using rhythmic features. Each student filled out the surveys both at the beginning (Pre) and the end of the semester (Post). To capture relationships between biobehavioral rhythms and changes in the mental health of students, we categorized students into five groups according to the survey measures for depression and loneliness. For simplicity of representation, we further label low loneliness and no depression categories as 1, and high loneliness and high depression as 2. The five mental health categories are as follows:
All students.
Pre1_Post1: not having a mental health condition in both pre-semester and post semester surveys.
Pre1_Post2: not having a mental health condition in the pre-semester survey, but having it in the post-semester survey.
Pre2_Post2: having a mental health condition in both surveys.
Pre2_Post1: having a mental health condition in the pre-semester survey, but not in the post-semester survey.

The following sections describe our observations and findings. To distinguish the mental health groups in the two conditions, we add an L and D to the mental health group for loneliness (e.g., L_Pre1_Post2) and depression (e.g., D_Pre1_Post2) respectively.

TABLE 1

| | TW1 | | | TW2 | | | TW3 | | | TW4 | | | TW5 | | | TW6 | | | TW7 | | | TW8 | | |
|---|---|---|---|---|---|---|---|---|---|---|---|---|---|---|---|---|---|---|---|---|---|---|---|---|
| Group | N | [symbol] | [symbol] | N | [symbol] | [symbol] | N | [symbol] | [symbol] | N | [symbol] | [symbol] | N | [symbol] | [symbol] | N | [symbol] | [symbol] | N | [symbol] | [symbol] | N | [symbol] | [symbol] |
| All | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |

[symbol]

indicates data missing or illegible when filed

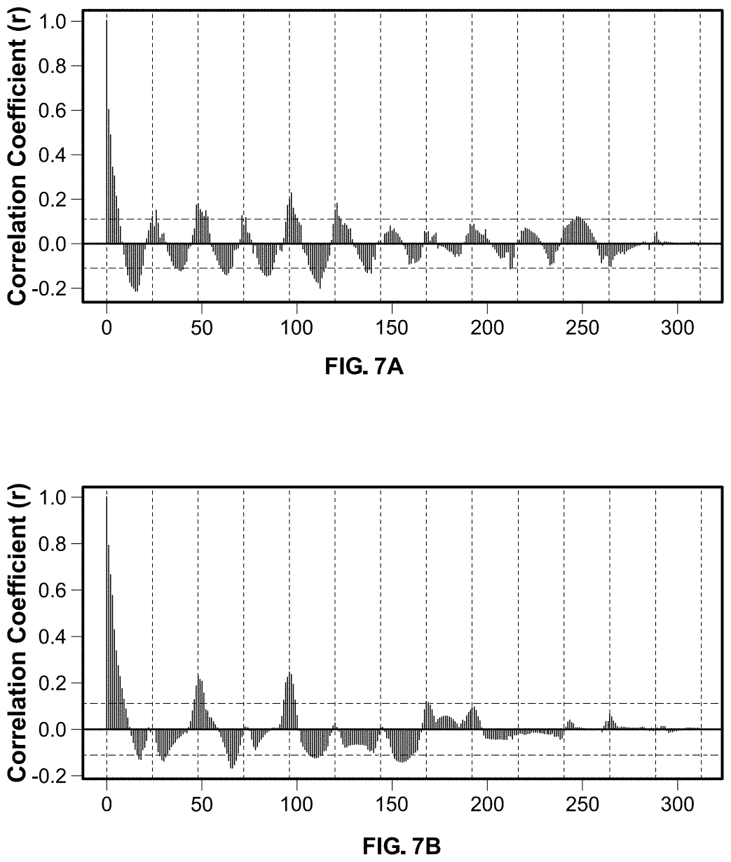

Detection of rhythmicity and regularity in student data. To investigate whether we can observe rhythmicity in data collected from students' smartphones and Fitbits (Question 1) and whether students' rhythms remains stable throughout the semester (Question 2), we used Autocorrelation and Periodogram to model students' rhythms in each time window for each sensor feature. FIG. 7 shows the correlogram of the number of restless sleep bouts in two students from different groups, one with low loneliness throughout the semester and the other with high loneliness at the end of the semester. FIG. 7 graphically illustrates correlograms of feature num_restless_bout (number of restless periods in sleep) in time window 4 for two students (FIG. 7(A): a student in L_Pre1_Post1, FIG. 7(B): a student in L_Pre1_Post2). The figure visually depicts differences in the rhythms of these two students where the correlogram belonging to student with high loneliness projects a less stable rhythm towards the end of time series. To further quantify such differences in cyclic rhythms of students, we apply Periodogram to 1) detect dominant periods in students' data and 2) measure variability in those periods among students with different health status.

Our results shows that the most dominant cyclic periods in each time window are 24- and 12-hours for all sensor features. For example, for sleep duration feature in depression category, this trend is consistent in all students regardless of the mental health condition where on average 97.6% and 69.6% of students have 24- and 12-hours as dominant periods in their data across time windows (Tables 1 and 2). Referring to Table 1, provided is the top two dominant periods of sleep duration feature for depression groups. N is the number of students in the group. P1 is the most dominant period (i.e., the percentage of students that have the period is highest among all periods). The percentage in parenthesis is the percentage of students with that period. P2 is the second dominant period. The percentages, however, have a declining trend starting from TW4 (around midterms) towards the end of the semester. This trend can be expected because of the increase in students' workload that cause irregularity in sleep duration. The lowest percentages across all time windows (46.3% on average) are observed in the 12-hour period of students in group D_Pre2 Post2, i.e., students who were depressed throughout the semester. In particular, there is no 12-hour period observed for this group in TW1 (the first two weeks) and TW8 (the last two weeks). The 12-hour or half-day period relates to diurnal/nocturnal activities and this trend may be indicative of higher irregularity in sleep behavior among students with depression throughout the semester especially at the beginning and towards the end of the semester. Our observations are consistent with other studies. It was observed that older adults with depression have lower sleep regularity index in a study of 138 participants [49]. It was observed that irregular sleepers showed more negative moods, including depression, in a study of male college students [60].

TABLE 2
Pre1_Post2

| | Loneliness | | | | Depression | | | |
|---|---|---|---|---|---|---|---|---|
| Time Window | N | P1 (%) | P2 (%) | P3 (%) | N | P1 (%) | P2 (%) | P3 (%) |
| TW1 | 17 | 24 (100) | 12 (71) | 312 (35) | 35 | 24 (100) | 12 (89) | 312 (34) |
| TW2 | 15 | 24 (93) | 12 (87) | 312 (40) | 34 | 24 (97) | 12 (88) | 312 (38) |
| TW3 | 16 | 21 (100) | 12 (88) | 156 (31) | 35 | 21 (91) | 12 (80) | 156 (31) |
| TW4 | 15 | 24 (73) | 12 (53) | 312 (33) | 33 | 24 (91) | 12 (64) | 78 (40) |
| TW5 | 14 | 24 (100) | 12 (64) | 156 (29) | 33 | 24 (97) | 12 (58) | 312 (36) |
| TW6 | 12 | 24 (92) | 12 (67) | 78 (33) | 33 | 24 (94) | 12 (64) | 78 (45) |
| TW7 | 13 | 24 (85) | 12 (54) | 156 (31) | 33 | 24 (91) | 12 (61) | 156 (10) |
| TW8 | 11 | 24 (91) | 12 (55) | 72 (45) | 23 | 24 (93) | 12 (78) | 72 (32) |

Referring to Table 2, provided is the top three dominant periods of sleep duration (minutes asleep) feature for Pre1_Post2 groups. N is the number of students in the group. P1 is the most dominant period (i.e., the percentage of students that have this period is highest among all periods). The percentage in parenthesis is the percentage of students that have the period. P2 and P3 are the second and third dominant periods.

We further analyzed changes in periodicity of sleep duration in students who started the semester with normal health status but developed depression or loneliness towards the end (D_Pre1_Post2 or L_Pre1_Post2). Table 2 shows that the dominant periods of 24- and 12-hours are preserved for the sleep duration feature in all time windows for both loneliness and depression groups. While the same declining trend towards the end of the semester exists for both loneliness and depression groups, a sharper slope is observed for the 12-hour period. The lowest percentage of students in this group with 24- and 12-hour periods are in time windows 4 and 5 with 73% in loneliness category (24-hour), 91% in depression category (24-hour), 53% in loneliness category (12-hour), and 57% in depression category (12-hour). Given that time windows 4 and 5 intersect with midterm and spring break, these observations points to changes in sleep patterns among students whose mental health worsens over the semester.

The third dominant periods for sleep duration across all time windows include 312-hour (13 days), 156-hour (6.5 days), and 78-hour (3.25 days). This is an interesting observation as these numbers are multiplies of the 78-hour period. In other words, it seems sleep duration of roughly one third of the population in these groups follow a weekly pattern that may be imposed by class schedules. Referring to Table 3, provided is the percentage of participants with 24-hour period across all sensor features.

TABLE 3
% of Participants with 24-hour period

| Audio | Battery | Bluetooth | Calorie | Location | Location Map | Call&Messages | Screen | Sleep | Steps | Wifi |
|---|---|---|---|---|---|---|---|---|---|---|
| 62 | 13 | 42 | 92 | 41 | 17 | 18 | 36 | 69 | 95 | 83 |

Overall and across all sensor features, we observe the 24-hour as the dominant period for over 52% of the student population with highest percentages belonging to steps (95%), calories (92%), wifi (83%), and sleep (68%). Table 3 presents the overall percentages for each sensor. Calories and steps relate to physical activity. The high percentage of students with 24-hour cycles in these two sensor categories is indicative of regular daily exercise and movement. While there is a low percentage of students with regularity in their cyclic location patterns and visited places (Location Map features), it seems a large number of students have regular daily patterns of using Wifi. This pattern could be expected given that the first-year students live in dorms and are mostly on campus. Interestingly, a low percentage of students seem to have regular cyclic patterns of phone usage (Screen, 36%; Call&Messages, 18%; Battery 13%). While phone use especially battery charging patterns are expected to be cyclic, (e.g., charging the phone at night), these observations present the possibility of different phone use behavior among students.

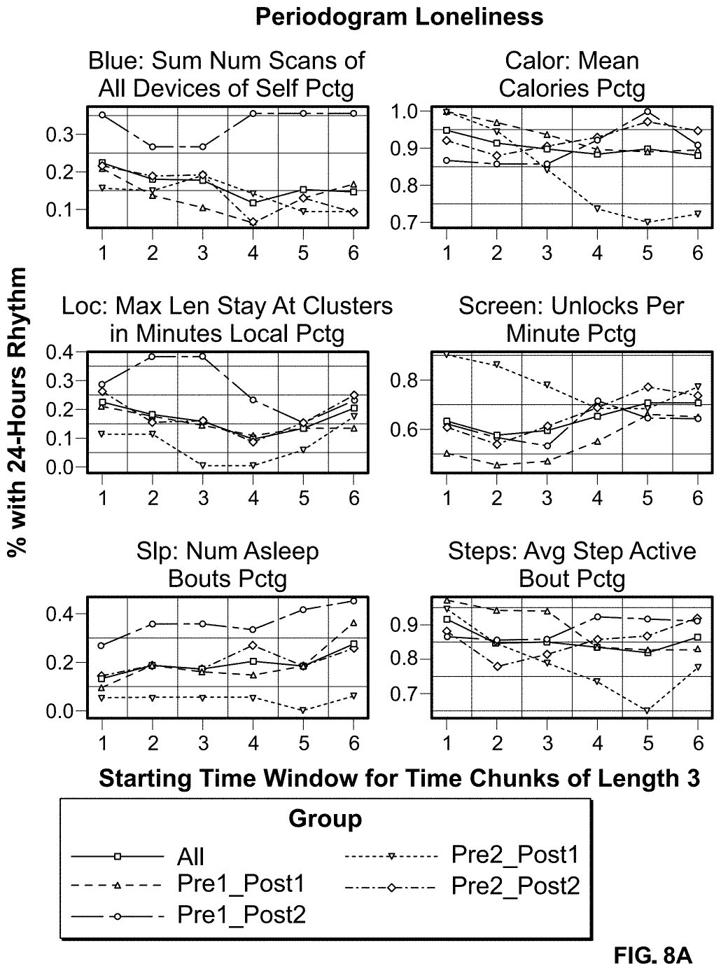

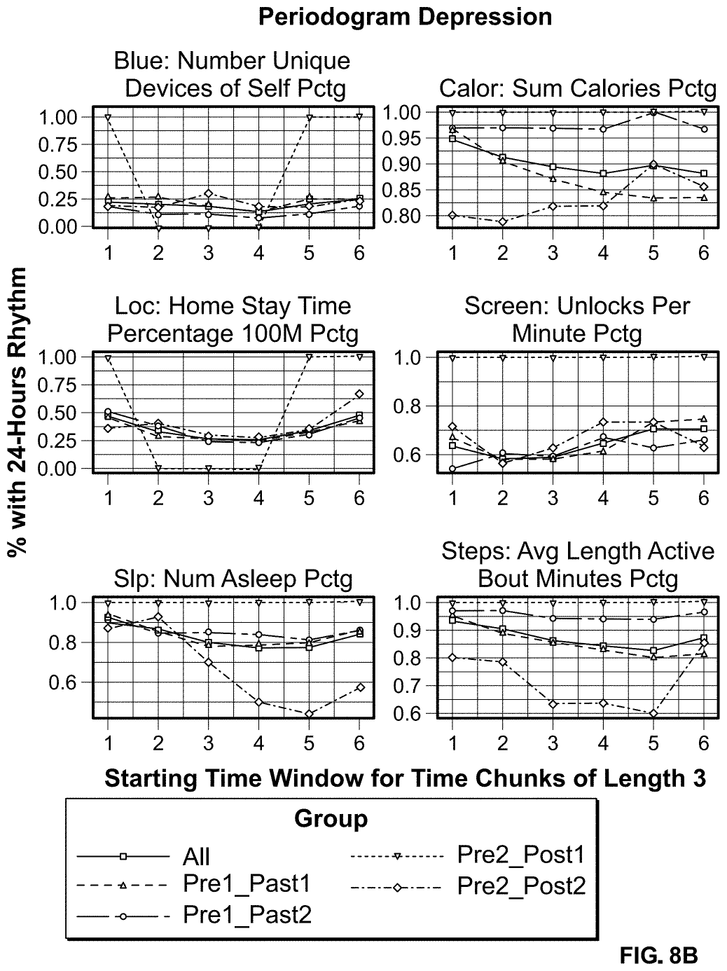

Following these observations, we further look at the percentage of participants in each mental health group that had 24-hours as one of their dominant rhythms for each time chunk. This would help observe the extent to which students preserved their normal circadian rhythm over the semester. Recall that time chunks consist of k consecutive time windows, there were 36 different time chunks in total for 8 time windows of length 2 in the dataset. In each time chunk, a participant had 24-hour as a dominant rhythm if and only if this participant had 24-hour as a dominant rhythm in all time windows in that time chunk. FIG. 8 graphically illustrates the percentage of participants with 24-hour as the dominant rhythm (y-axis) in each mental health group for each time chunk of length 3 (x-axis). We chose one representative feature from each sensor stream, i.e., bluetooth (abbreviated as short-long dash [pre1_past2] in the figure), location (loc), sleep (slp), calories (calor), screen, and steps for further analysis. Turning to FIG. 8, FIG. 8 graphically illustrates the plots show the percentage of participants with 24-hour as the dominant rhythm (y-axis) in each mental health group (FIG. 8(A): loneliness, FIG. 8(B): depression) for each time chunk of length 3 (x-axis). The data point at x=i corresponds to the time chunk of length 3 starting at tw (i.e., tc₃ᵢ). It represents the percentage of participants with 24-hour as the dominant rhythm in all the 3 time windows twᵢ, twᵢ₊₁, twᵢ₊₂.

For loneliness, the group with low loneliness at the beginning and high loneliness at the end of the semester (L_Pre1_Post2) shows an overall higher percentage of 24-hour rhythms for features of sleep, location, and bluetooth across time windows. The opposite group with high loneliness at the beginning and low loneliness at the end of the semester (L_Pre2_Post1) shows lower percentage of 24-hour rhythms for features of calories and steps but higher percentages for screen features. The bluetooth feature in the top left of FIG. 8(A) which represents the cyclic patterns of the scanned devices belonging to the person is a proxy of social isolation, i.e., the person not being around other people (and their devices) and being mostly by themselves. Starting from TW3 (week 3, 4 and 5), the percentage of students with regular daily cycle for this features in L_Pre1_Post2 and L_Pre2_Post1 groups sharply increase and decrease respectively. In other words, while more students with low loneliness at the beginning and high loneliness at the end of the semester start having a regular social isolation patterns on a daily basis towards the end of the semester, fewer students in the opposite group with high loneliness at the beginning and low loneliness at the end of the semester experience this trend. A very similar pattern is observed for another socially relevant feature, namely the length of stay in significant locations. The trend is relatively stable and slightly decreasing in students with no loneliness which reflects stability of behavior in this group. For sleep, steps and calorie burn, we observe an almost counter intuitive opposite cyclic behavior among L_Pre1_Post2 and L_Pre2_Post1 groups. It seems more students with loneliness toward the end of semester engage in regular physical activities as projected by calories and steps features and have more regular sleep duration cycles. A relatively similar behavior is observed for the burned calories feature in depression groups (FIG. 8 (B) top right). While regularity in physical activities slightly increases in students with depression (D_Pre2_Post2), it appears to decrease in students with no depression (D_Pre1_Post1) across time windows. While existing studies, e.g., [7, 19, 59] point to negative associations of physical activities and mental health, we believe increase in regular physical activities towards the end of the semester may be a coping attempt by students with mental health problems.

But trends generally look different for depression groups in FIG. 8 (B). All groups except D_Pre2_Post1 had similar percentage of regular 24- and 12-hour periods for bluetooth, location and screen across time windows. Since there is only one participant in group D_Pre2_Post1, we exclude it from further discussion. While the group with no depression at the beginning and with depression at the end of the semester (D_Pre1_Post2) shows highest percentage of normal 24-hour rhythms for features of calories and steps across all time windows, the group that was depressed throughout the semester (D_Pre2_Post2) shows lowest percentages for steps, sleep and calories. In particular, regularity of sleep in these students seems to decline drastically across time windows. Although expected, this sharp trend is a valuable observation for further exploration of relationships between change in sleep cycles and depression status. In a previous study [49] it was also observed that sleep irregularity is indicative of depression, but no existing study has analyzed the relationship between change in sleep cycles and change in depression status. Our observations provide new findings and insights that call for further and more rigorous investigations.

Prediction of Mental Health Status with Rhythmic Features. The third and fourth questions in our analysis relate to the feasibility of using parameters of biobehavioral rhythms to predict mental health status in students. In our framework, we utilize dominant periods detected from the previous step using Periodogram to build Cosinor models of biobehavioral data. This process generates rhythmic features that are fed into the machine learning process to classify post-semester loneliness and depression categories (low loneliness vs. high loneliness and no depression vs. with depression) of the students. We build two types of datasets one with single sensors only and one with multiple sensors.

For Single Sensor datasets, we use the rhythmic features of each sensor feature separately, i.e., for each sensor feature and each time chunk (with time windows of two weeks), we take the rhythmic features of this sensor feature and time chunk to form the input dataset. We remove datasets with more than 30% missing instances (80 training instances) as we consider it too small to generate a reliable and generalizable model. For Multiple Sensors datasets, we select the sensor features that provide accuracy above baseline in models built with single sensors. For both approaches, we use the majority class ratio i.e., the category that has the highest percentage of labels for that category as the comparison baseline. We then repeat the same process we followed for single sensor datasets but this time for the combination of sensor features, i.e., for each time chunk and each combination of sensors, we take the rhythmic features of the selected sensor features of those sensors and time chunk to form the input dataset. Other than the difference in input dataset, the machine learning pipeline is the same for the two types of datasets.

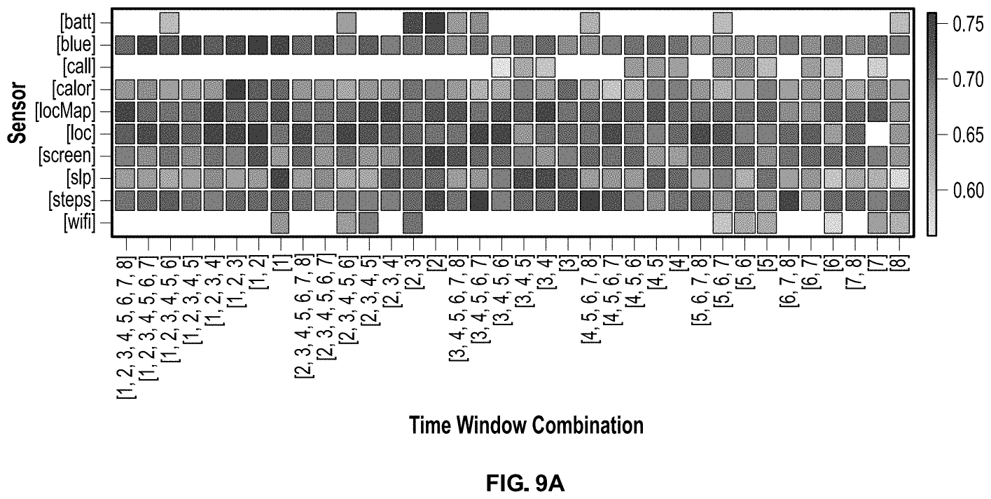

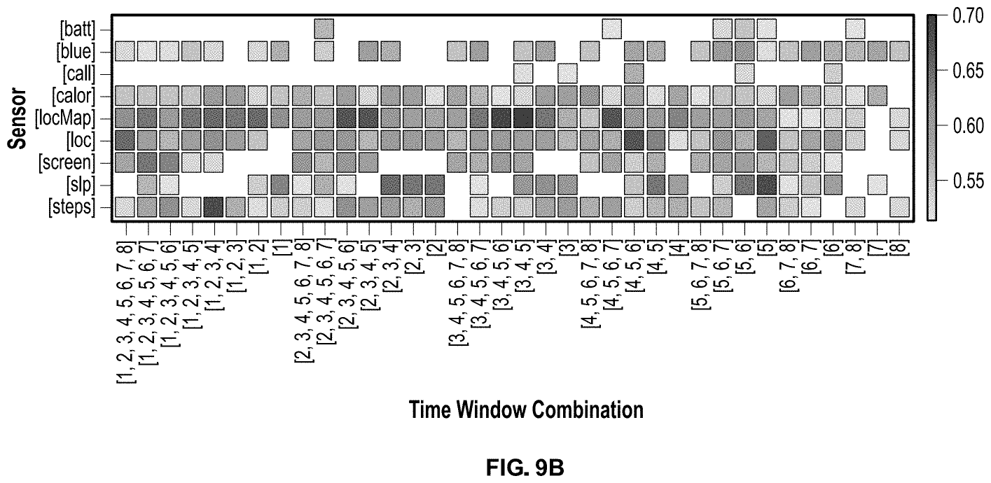

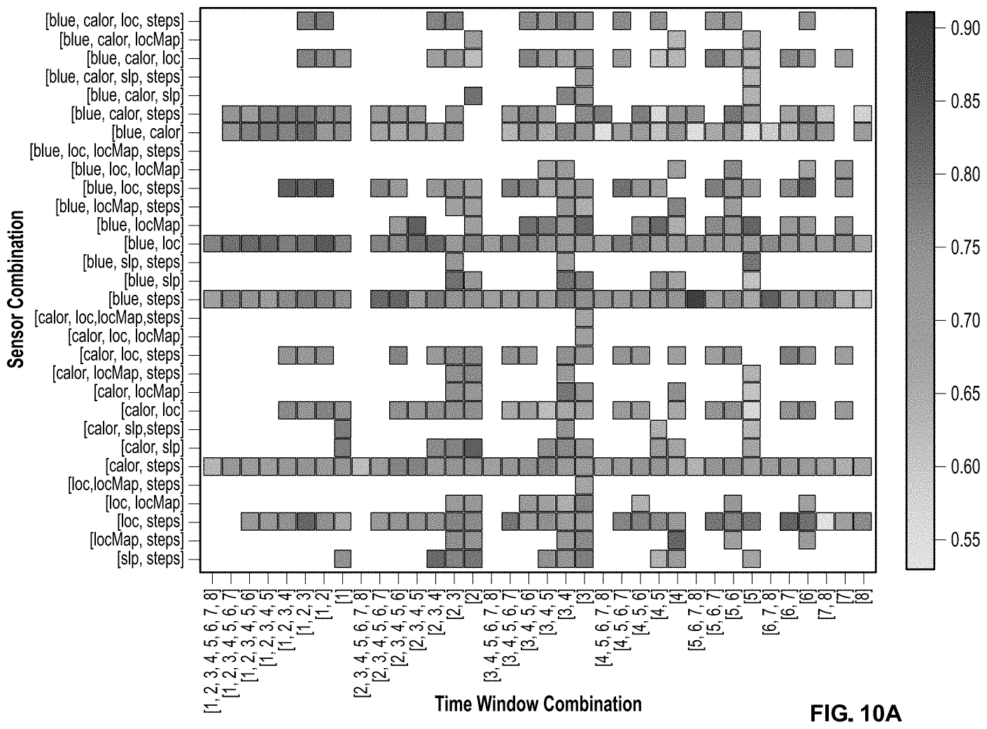

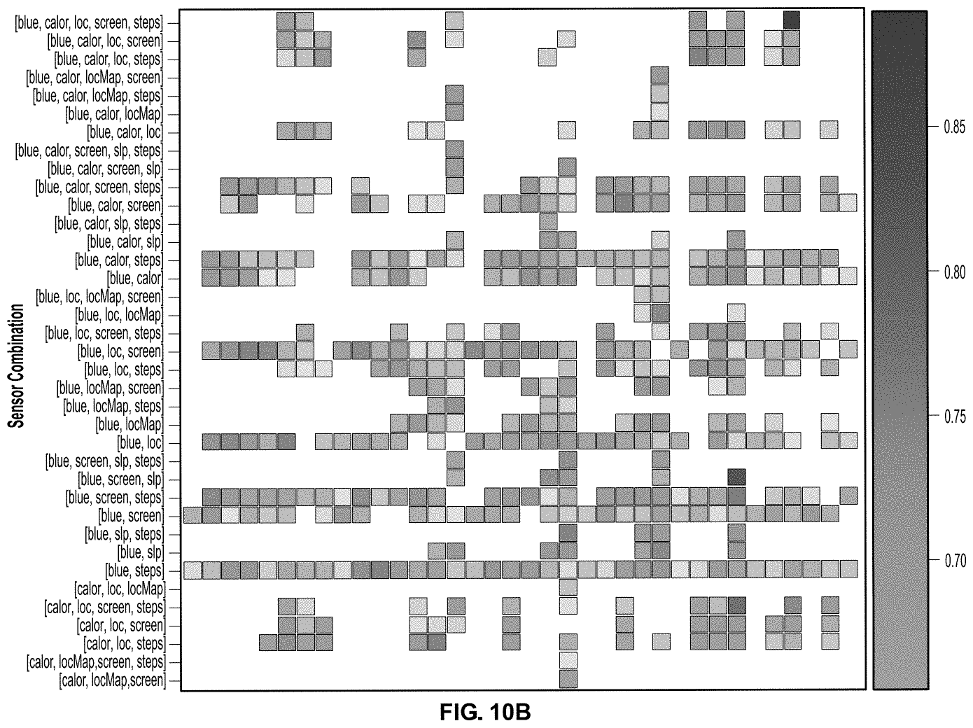

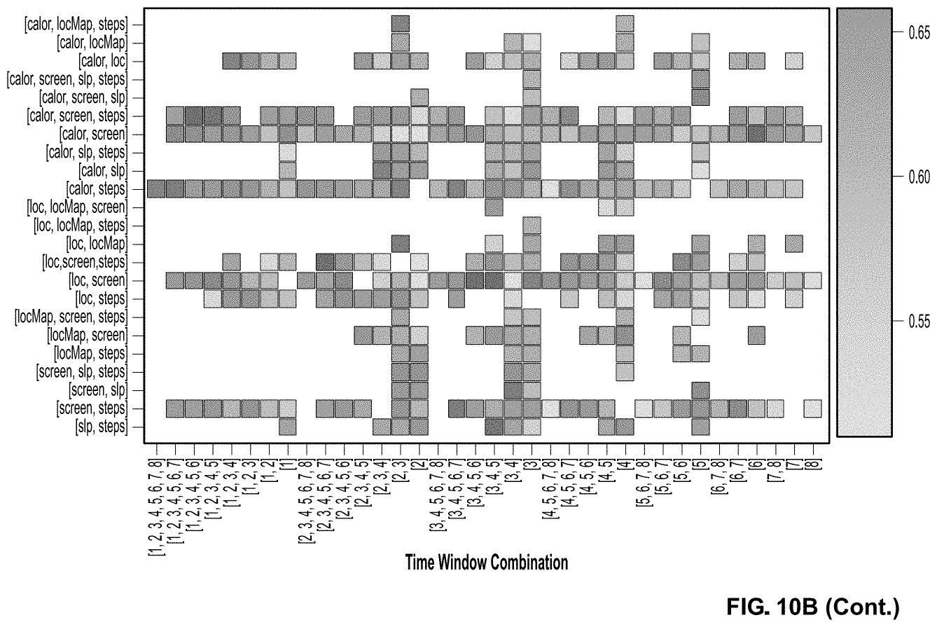

Given the imbalanced datasets for both health conditions i.e., different number of samples in the two classes (e.g., 59% of samples in category 1 vs. 41% in category 2 of depression), using the accuracy will not be adequate for performance evaluation and needs to be accompanied by other measures such as F1. For every combination of time window and sensor, the F1 score is used to select the model with the best performance. We build models with single sensor and multiple sensors datasets for both mental health conditions. The results of all combinations are shown in FIGS. 9 and 10. The heatmaps use the depth of color to represent the F1 score. Given the large number of features, we only report results with accuracy above the baseline (majority class percentage). Through the single sensor modeling, we can judge which type of sensor is most effective in predicting mental health. Overall, we find that the models with multiple sensors improve the prediction performance. A summarization of the results are listed in Table 4.

Single Sensor Modeling. The F1 scores of machine learning models with single sensor features are shown in FIGS. 9(A) and (B) for loneliness and depression, respectfully. FIG. 9 graphically illustrates the heatmap displays the largest F1 score in the loneliness (FIG. 9(A)) prediction model and depression (FIG. 9(B)) prediction model trained by a combination of different single sensor features and time windows. Rhythm parameters obtained from Cosinor models built for features related to bluetooth, calories, location, sleep, and steps perform better in predicting both loneliness and depression levels. Overall, the models for loneliness prediction obtain higher accuracy (F1) scores than depression models (Table 4) which may be due to more sparsity in depression datasets. Although the best model to classify post-semester loneliness is built using Gradient Boosting on rhythm parameters of calorie data from tw₁ to tw₃ with an F1 score of 0.76, more models built on rhythms of location and locationMap provide high performance. The best model for post-semester depression with an F1 score of 0.7 is also built using Gradient Boosting but on the locationMap data from tw₃ to tw₅. Compared to other sensors, models using rhythmic parameters from locationMap features show better performance for predicting post-semester depression (six out of ten models with the highest F1 score use locationMap features). Although the F1 scores of models with a single time window are generally lower than models with multiple time windows, there are some exceptions in the heatmaps of both loneliness and depression. For example, the loneliness model using sleep features in tw1 achieves an F1 score of 0.75, and the F1 score of the depression model using sleep features in tw₅ equals 0.68. Interestingly and somewhat counter-intuitively, across all sensors, the majority of models (avg. 57.5% for single sensors and 53.5% for multiple sensors) using early semester time windows (tw₁ to tw₄) appear to have higher F1 scores for post-semester loneliness and depression prediction than late semester time windows. We believe this observation provides initial evidence for the possibility of early detection of mental health status via monitoring of changes in biobehavioral rhythms.

Multiple Sensor Modeling. We do the same analysis for the combination of sensor features. From FIGS. 10(A) and (B), we observe that the combination of multiple sensor features contributes to the improvement of F1 score for loneliness and depression, respectfully. Referring to FIG. 10(A), the heatmap displays the largest F1 score in the loneliness prediction model trained by a combination of different multiple sensor features and time windows. For example, the combinations related to steps, sleep, location, calorie, and Bluetooth end with better results. For predicting loneliness, the best model is built with Logistic Regression, which uses the Bluetooth and steps data from tw₅ to tw₈ and obtains an F1 score of 0.91. Turning to FIG. 10(B), for predicting depression, the best model is obtained from Logistic Regression using the rhythm parameters from Bluetooth, calorie, location, screen, and steps features. The model only uses tw₆ to predict depression with an F1 score of 0.89. The best model predicting depression (FIG. 10(B)) has a lower F1 score than the best model predicting loneliness (FIG. 10(A)), which is the same as the single sensor model and may be due to sparsity in sensor data.

Table 4 summarizes the mean and max of F1 scores for models built with each combination of the feature selection and machine learning methods. Referring to Table 4, provided is the summary of the mean and maximal values of F1 scores for each combination of feature selection and machine learning methods shown in the heatmaps 7, 8. The bold values are either the biggest mean value of F1 scores, or the biggest maximal values of F1 scores. In single sensor modeling, the combinations of Logistic Regression with Lasso and Randomized Logistic Regression) perform best for predicting loneliness with the mean and max F1 score of 0.7 and 0.76 respectively. The combination of Gradient Boosting and Information Gain provides the highest F1 score for prediction of depression. For the multiple sensor modeling, we observe that the maximum F1 scores of predicting loneliness and depression are 0.91 and 0.89, which are obtained from the combination of Logistic Regression and Lasso. Overall, for the majority of approaches, the combination of Gradient Boosting and Information Gain provides the best performance. This combination should be further evaluated with other similar datasets to replicate and confirm their superior performance over other algorithm combinations.

Dominant rhythm parameters that predict mental health. Although we used three feature selection methods in our evaluation, we observed that the Information Gain method provided more reliable and complete list of features during the training. Table 5 shows the rhythm features that are selected most frequently by Information Gain during depression prediction for each sensor feature in each time window. Referring to the Table 5, provided is the most frequently selected rhythm features by Information Gain during depression prediction. The vertical dominant feature (VDominant) is the most commonly selected feature for most of the sensors at a given time window, and the horizontal dominant feature (HDominant) is the most commonly selected feature in most time windows for a given sensor. The overall dominant feature (the feature at the bottom right corner in bold font) is the most commonly selected feature for all sensors and time windows. If two features are the most commonly selected features for the same number of sensors/time windows, we break the tie by taking the feature with higher frequency. Overall, Orthophase is selected most frequently for all sensors and time windows. Magnitude comes the second. Given that Phase and Magnitude reflect duration and intensity of biobehavioral features, frequent selection of these parameters suggest an important relationship with mental health status.

TABLE 4

| | Single Sensor | | | | | | Multiple sensors | | | | | |
|---|---|---|---|---|---|---|---|---|---|---|---|---|
| | Loneliness mean(max) | | | Depression mean(max) | | | Loneliness mean(max) | | | Depression mean(max) | | |
| | GB | LR | RF | GB | LR | RF | GB | LR | RF | GB | LR | RF |
| IG | 0.69 (0.76) | 0.69 (0.76) | 0.66 (0.72) | 0.58 (0.70) | 0.60 (0.61) | 0.56 (0.63) | 0.73 (0.83) | 0.72 (0.78) | 0.69 (0.81) | 0.96 (0.83) | 0.60 (0.66) | 0.63 (0.76) |
| Lasso | 0.68 (0.72) | 0.70 (0.76) | 0.74 (0.74) | 0.57 (0.68) | 0.57 (0.64) | 0.55 (0.59) | 0.72 (0.78) | 0.75 (0.91) | | 0.59 (0.66) | 0.67 (0.89) | 0.54 (0.54) |
| RLR | | 0.70 (0.76) | 0.68 (0.73) | 0.58 (0.65) | 0.56 (0.65) | 0.57 (0.60) | 0.75 (0.81) | 0.73 (0.82) | 0.76 (0.84) | 0.65 (0.78) | 0.65 (0.79) | 0.65 (0.79) |

In addition to main rhythmic features, i.e., Mesor, Amplitude/Magnitude, and Ortho/Bathyphase, we observe frequent selection of features related to the fit of Cosinor models including the significance level of the fit (P), Standard Errors (SE) and Percent Rhythm (PR and IPR), i.e. the proportion of the overall variance accounted for by the fitted model. Higher levels of these parameters reflect higher variation in data, and therefore, frequent selection of these parameters indicates the power of regularity/irregularity of biobehavioral rhythms in predicting mental health status.

TABLE 5

| | TW1 | TW2 | TW3 | TW4 | TW5 | TW6 | TW7 | TW8 | HDominant |
|---|---|---|---|---|---|---|---|---|---|
| Audio | Amp SE | Mesor SE | Amp SE | IPR | Magnitude | Amp SE | Bathyphase | P | Amp SE |
| Battery | IPR | PR | Mesor SE | Mesor SE | Orthophase | Magnitude | Orthophase | Bathyphase | Mesor SE |
| Bluetooth | Magnitude | Bathyphase | Amp | P | IPR | Orthophase | Mesor SE | Orthophase | Orthophase |
| Call | IPR | PHI | IPR | IPR | Amp SE | Bathyphase | Orthophase | Magnitude | IPR |
| Calorie | Mesor | Magnitude | Magnitude | Bathyphase | Orthophase | Orthophase | IPR | Magnitude | Magnitude |
| Location | PHI SE | Magnitude | Mesor | PR | IPR | Mesor | Amp SE | IPR | Mesor |
| Location Map | Orthophase | Magnitude | Mesor | Orthophase | PHI | Bathyphase | Orthophase | Bathyphase | Orthophase |
| Messages | Orthophase | Magnitude | LCM | PR | Mesor SE | Bathyphase | PHI SE | Magnitude | Magnitude |
| Screen | Amp | P | Orthophase | Orthophase | PR | Orthophase | IP | Amp SE | Orthophase |
| Sleep | Bathyphase | PHI SE | Mesor | Orthophase | PHI SE | IP | PHI SE | Bathyphase | Bathyphase |
| Steps | P | Orthophase | Magnitude | Bathyphase | PR | IPR | IPR | Magnitude | Magnitude |
| Wifi | Amp | Mesor SE | Mesor | Orthophase | Magnitude | IPR | IP | Amp SE | Magnitude |
| VDominant | Amp | Magnitude | Mesor | Orthophase | Orthophase | Bathyphase | Orthophase | Magnitude | Orthophase |

Comparison with Models Built without Rhythm Parameters. To better understand the capability of our framework in utilizing rhythmic features to predict an outcome, we compare the prediction performance of the models with rhythm modeling against the models without rhythm modeling. Specifically, we select the best performing sensor feature in each time window, run exactly the same machine learning pipeline on the raw feature data without rhythm modeling, and compute the F1 score. Table 6 shows that the pipeline with rhythm modeling outperforms the one without by a large margin on most of the features. This observation is consistent for both loneliness and depression predictions. Referring to Table 6, provided is the F1 of machine learning models with rhythm modeling (rhythm) and without rhythm modeling (raw features). Left: Loneliness; Right: Depression.

TABLE 6

| Time Window | Feature | Rhythm-F1 | Raw-F1 |
|---|---|---|---|
| 1 | shortest period spent at Halls | 0.66 | 0.54 |
| 2 | longest awake period length | 0.64 | 0.49 |
| 3 | number of awakes | 0.63 | 0.47 |
| 4 | maximum calories increase between 5-min periods | 0.66 | 0.60 |
| 5 | shortest alseep period length | 0.70 | 0.69 |
| 6 | total distance traveled | 0.65 | 0.50 |
| 7 | maximum calories decrease between 5-min periods | 0.67 | 0.59 |
| 8 | minutes spent at Halls | 0.65 | 0.62 |
| 1 | shortest period spent at Halls | 0.69 | 0.55 |
| 2 | longest awake period length | 0.67 | 0.47 |
| 3 | total alseep time | 0.67 | 0.49 |
| 4 | number of awakes | 0.62 | 0.56 |
| 5 | percentage of time spent moving | 0.72 | 0.52 |
| 6 | longest period spent at athletic areas | 0.68 | 0.43 |
| 7 | total change of calories | 0.68 | 0.53 |
| 8 | variance of moving speed | 0.67 | 0.48 |

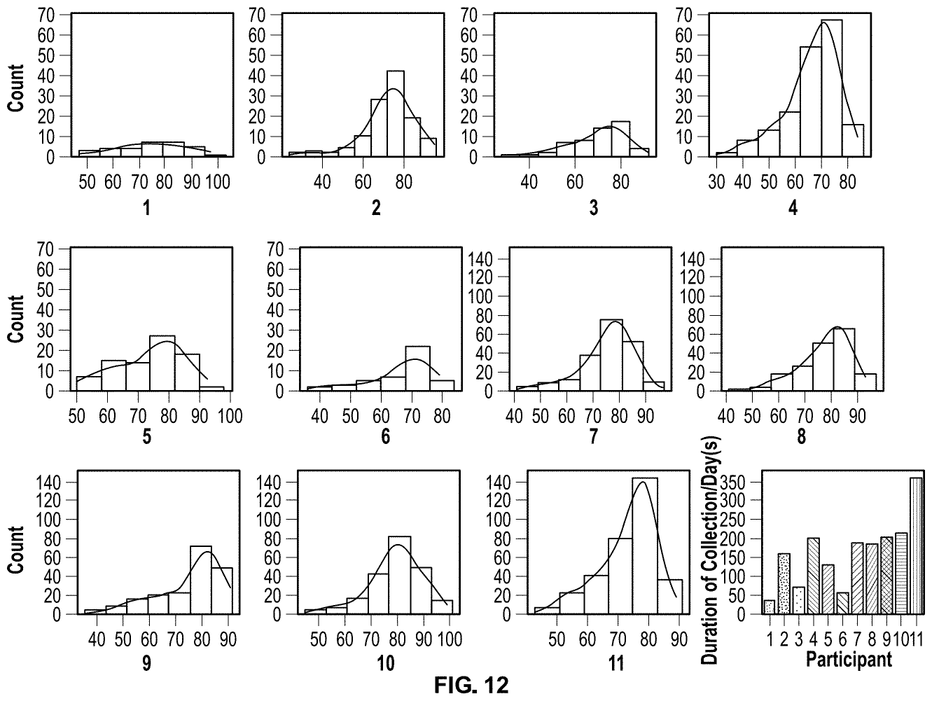

Case 2: Biobehavioral Rhythm Modeling for Readiness Prediction Using Data from OURA Ring. We chose a second dataset to evaluate the framework's flexibility in modeling various types of data and applying different analysis approaches. For this case, we used data from 11 volunteers who continuously wore Oura ring, a smart and convenient health tracker for several months. As shown in the last plot of FIG. 12, the length of data collection varies per participant and ranges from 31 to 323 days. The long-term data makes it possible to detect and observe rhythms with larger cyclic periods than a day, e.g. weeks or months. As such, we design our analysis to answer the following:
(1) Are there common cycles in participants' data per sensor and across sensors, and can we identify similarities and differences in cyclic periods among participants despite differences in the length of their data?
(2) How accurately can individual rhythm models per sensor feature and per participant predict average readiness?

Physiological Data Processing. OURA collects sleep, heart rate, skin temperature, calories, steps, and activity. Sleep, heart rate, and skin temperature samples are collected every five minutes during night hours; and activity, calories, and steps are sampled every 5 minutes during the day. The data is summarized and stored on the OURA cloud platform. As our goal is to detect cycles with multiple-day lengths, we aggregate the features into daily intervals (as opposed to the previous case that used hours). In total, we use 31 features such as total duration of sleep, lowest/average heart rate, average metabolism level, total amount of calories burned, and total number of steps during the day. To be able to detect longest periods in participants' data, we refrain from segmenting data into common time windows and use the entire time series data for the analysis. The convenience of wearing the ring and its long battery life leads to good quality data with low missing rates (Max 15.6% in our data). We use the moving average method to handle the missing values.

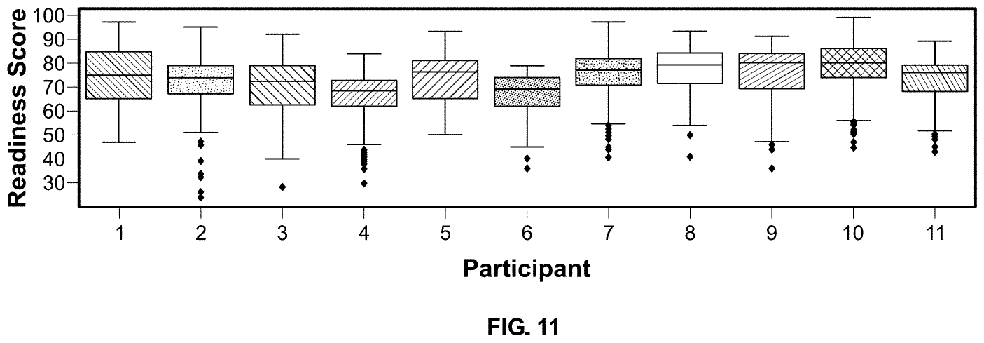

Readiness Score as Ground Truth. Besides the physiological features, Oura provides a readiness score, i.e. an evaluation of body's overall recovery rate after waking up in the morning. The readiness score ranges from 0 to 100 with scores over 85 indicating high readiness for challenging tasks and scores below 70 indicating poor body state and need for recovery. In our dataset, participants' readiness scores range from 24 to 99 with an average score of 74, and standard deviation of 11.4. FIGS. 12 and 11 show the distribution and variation of daily readiness score for each participant. We calculate the average daily readiness score for each participant and use it as ground truth to explore how well we can use the rhythms to predict the readiness score. FIG. 11 graphically illustrates the 1 to 11 boxplots display the minimum, median, maximum, and quartile of the daily readiness scores for each participant. Most daily readiness scores are clustered in the range from 70 to 85. FIG. 12 graphically illustrates the histograms from 1 to 11 display the distribution of the daily readiness scores for each participant, and the last bar plot shows the duration of each participant's data collection.

Detection of cycles in OURA-Ring Data. Our first analysis questions relate to detection of common cycles in participants' data and in the physiological sensors. To detect significant periods, we apply Periodogram on the time series data of each sensor feature per participant. In Tables 7 and 8, we list the most frequently detected periods of sensor features and summarize them by sensor type and participants. Referring to Table 7, provided is the dominant frequent periods for each sensor. The percentage in parenthesis is the percentage of participants with the significant period. Referring to Table 8, provided is the most frequent periods of all sensor features for each participant. The percentage in parenthesis is the percentage of sensor features with that period. The number 7 and its multiple 14 as well as its close preceding and following numbers of 6 and 8 appear most frequently in both tables suggesting near-weekly biobehavioral patterns. In particular, periods of Activity, Sleep, and Heart rate project near-weekly cycles across all participants. For example, Activity cycles of 6, 7, and 8 days are observed in 45%, 55%, and 36% of participants respectively. These cycles are also observed in sensor data of seven participants (63%). Calorie and Steps share periods of 2, 10, and 11 days with similar percentages. Although the percentages of participants with these cycles are low likely due to different movement patterns among participants, the common periods of these two sensors may be indicative of exercise cycles in those participants.

TABLE 7

| Sensor | Detected Period (% of Participants) |
|---|---|
| Activity | 7 (55), 2 (45), 6 (45), 8 (36), 4 (36) |
| Calorie | 2 (18), 11 (18), 10 (18), 4 (9), 81 (9), 20 (9) |
| Heart Rate | 7 (36), 27 (27), 8 (27), 14 (18), 18 (18) |
| Sleep | 8 (55), 3 (55), 7 (45), 6 (45), 11 (36) |
| Steps | 11 (27), 10 (27), 2 (18), 54 (18), 7 (18) |
| Skin | 12 (36), 14 (36), 15 (27), 27 (27), 34 (18) |
| Temperature | |

TABLE 8

| Participant | Detected Period (% of Sensor Features) |
|---|---|
| 1 | 7 (29), 34 (26), 2 (23), 3 (16), 39 (10) |
| 2 | 80 (42), 81 (39), 40 (35), 11 (29), 32 (26) |
| 3 | 77 (32), 10 (29), 24 (23), 7 (23), 26 (16) |
| 4 | 7 (52), 202 (39), 101 (35), 67 (19), 201 (16) |
| 5 | 66 (39), 65 (35), 130 (26), 8 (26), 26 (23) |
| 6 | 6 (35), 56 (29), 14 (23), 28 (13), 19 (10) |
| 7 | 31 (26), 11 (23), 190 (23), 95 (23), 38 (19) |
| 8 | 94 (42), 188 (29), 63 (29), 7 (23), 189 (23) |
| 9 | 68 (45), 102 (35), 29 (29), 204 (26), 41 (16) |
| 10 | 54 (45), 108 (39), 43 (35), 27 (23), 217 (32) |
| 11 | 126 (35), 42 (26), 28 (23), 5 (16), 7 (16) |

Prediction of Readiness with Rhythmic Features. For each participant, we use the three most significant periods identified by the Periodogram as input to the Cosinor method to build rhythm models per sensor feature. The rhythmic features are then entered in the machine learning process to predict average readiness per participant. Since the readiness score is a continuous variable, we build regression models to make predictions. Our choice of machine learning algorithms include Random Forest and Gradient Boosting with Information Gain and Lasso as feature selection methods. Similar to case 1 in mental health, we build models with single and multiple sensor combinations in a leave-one-participant-out cross validation, but instead of accuracy, we use the Root Mean Square Error (RMSE) as performance measure.

Table 9 lists the best RMSE achieved by single sensor models along with the most frequently selected features. Referring to Table 9, provided is the lowest RMSE of single sensor features and frequent rhythmic features selected by IG and Lasso. Among single sensor models, the model built with rhythmic feature of sleep data with an RMSE of 4.08 is a stronger predictor of readiness than others. In comparison, the combination of sleep, calories, and steps obtain an RMSE of 3.54, the lowest RMSE among all multiple sensor models, as shown in Table 10. Referring to Table 10, provided is the RMSE of multiple sensor models and frequent rhythmic features selected by those models. This combination takes into account both the activity of the human body during the day (calories) and the sleep quality at night (sleep). These observations are expected and confirm the impact of both sleep and physical activity on daily functioning of the body. Interestingly but not surprisingly, the frequently selected features across all sensors are standard errors of the rhythm parameters (i.e., PHI SE, MESOR SE, and Amp SE) as well as percent rhythm (PR) all of which are indicative of variation in the actual data. MESOR SE is the most dominant feature among both single and multiple sensor models. These results suggest that the level of variability and potentially irregularity in biobehavior may be most predictive of fluctuations in readiness.

Tables 9 and 10 also summarize the RMSE for models using each combination of feature selection and machine learning methods. The Gradient Boosting model with Lasso regression achieves the best performance for both single sensor and multiple sensor modeling, with an RMSE of 3.54. Using the same prediction model, the Information Gain performs better in single sensor modeling, and the results are reversed in multiple sensor modeling.

TABLE 9

| Sensor | Activity | | Calorie | | HR | | Sleep | | Step | | Skin Temperature | |
|---|---|---|---|---|---|---|---|---|---|---|---|---|
| Feature Selection | IG | Lasso | IG | Lasso | IG | Lasso | IG | Lasso | IG | Lasso | IG | Lasso |
| RMSE (GB) | 5.04 | 8.42 | 4.79 | 5.18 | 4.54 | 5.50 | 4.08 | 5.54 | 4.71 | 6.77 | 5.34 | 6.77 |
| RMSE (RF) | 5.25 | 8.52 | 4.38 | 4.51 | 4.65 | 6.20 | 4.20 | 5.68 | 4.81 | 7.30 | 5.48 | 7.30 |
| Frequent Rhyth [symbol] | PR | PHI | Mesor SE, | PHI | PR | PHI, | PR | P | Mesor SE, | Mesor, | Mesor SE, | PHI |
| Features | | | Amp SE | | | PHI,SE, P | | | Amp SE | P | Amp SE, P | |

[symbol]

indicates data missing or illegible when filed

TABLE 10

| Feature Selection | IG | Lasso |
|---|---|---|
| Sensor | sleep, calorie, step | sleep, calorie, step |
| RMSE (GB) | 3.73 | 3.54 |
| RMSE (RF) | 3.80 | 3.68 |
| Frequent Rhtyhmic | MESOR SE | MESOR |
| Features | | |

Discussion. An aspect of an embodiment of the present invention overcomes, among other things, several challenges in processing and modeling biobehavioral time series data from mobile and wearable devices that motivated the development of our novel computational framework. These challenges include, but are not limited thereto, 1) automated handling and processing of massive multimodal sensor data, 2) granular and fine-grained exploration of all signals to extract knowledge about biobehavioral cycles, and 3) computational steps for modeling, discovering, and quantification of common patterns.

An aspect of an embodiment of the present invention included, among other things, two case studies using different datasets, sensors, populations, and prediction tasks to demonstrate capabilities of our proposed computational framework in addressing the aforementioned challenges. Both cases demonstrated the ability of the framework to automatically process longitudinal multimodal sensor mobile data; extract fine-grained and granular features; detect periodicity in the data and use it to study rhythm stability and variation over time; build micro-rhythm models for each biobehavioral feature; and use those models in incorporate different analytic approaches to predict various health outcomes. We were able to build massive prediction models for both single sensors and different combination of sensors and to compare the results. We observed that the combination of multiple sensor features contributed to the improvement of prediction results. We also showed that the models built with rhythmic features outperform models build with the raw sensor features further demonstrating the feasibility of biobehavioral rhythms in prediction tasks.

Although some of our primary goals were to showcase capabilities and flexibility of the framework, our analyses also provided interesting and novel observations some of which can be used as initial evidence for further investigation. For example, although we used different datasets and population groups in case 1 and 2, we observed near-weekly sleep cycles in both populations. We also observed drastic decline in sleep duration cycles of depressed students throughout the semester. Even though existing research has repeatedly shown relationships between sleep and mental health, we believe our observation is unique in identifying relationships between change in cyclic patterns of sleep and mental health status. Our micro machine learning models of sensor features provided evidence that changes in biobehavioral rhythms in early weeks of the semester were predictive of post-semester depression and loneliness. This finding suggests monitoring biobehavioral rhythms may serve as useful tool for early prediction of change in mental health status. We also observed that rhythmic parameters of Phase and Magnitude that reflect duration and intensity of biobehavioral features as well as parameters related to variability in the cyclic time series models (e.g., SEs and PR) were frequently selected in the machine learning process indicating the power of the intensity, duration, and regularity/irregularity of biobehavioral rhythms in the prediction of health outcomes. Since there is no comparable study in biobehavioral rhythms for prediction of health and wellness, we only compared our observations with closest studies of loneliness and depression. We submit that our initial findings open up for more studies using our framework to replicate the results.

One of the central themes of this disclosure was introducing the computational framework and its main functionality. However, the framework is generalizable and can be adapted and extended to include more functionalities and features. The advancements include 1) adding more data sources such as weather, environment, work schedules, and social engagements to draw a more holistic picture of biobehavioral rhythms in individuals and groups of people, 2) adding a conclusive set of periodic functions and methods with diverse characteristics that provide the possibility of uncovering different cyclic aspects in data, 3) developing novel methods for measuring stability of rhythms, and 4) advancing the machine learning component to incorporate a comprehensive selection of analytic methods that further enhances the capabilities of the framework to be used for predictive modeling of cyclic biobehavior.

For the current implementation, we limited our periodic functions to Autocorrelation, Periodogram, and Cosinor. In other embodiments, we expect to build an ensemble system incorporating different types of rhythm detection algorithms, and design a voting algorithm to aggregate the outputs of period detection algorithms. For example, the most frequent detected period by various detection algorithms will be treated as the dominant period. We also plan to extend the framework by adding and evaluating novel methods to quantify collective stability of individual and group rhythms.

### EXAMPLES

Practice of an aspect of an embodiment (or embodiments) of the invention will be still more fully understood from the following examples and experimental results, which are presented herein for illustration only and should not be construed as limiting the invention in any way.

Example 1. A computer-implemented method for modeling biobehavioral rhythms of a subject. The method may comprise: receiving sensor data collected from a mobile device and/or wearable device; extracting specified sensor features from said received sensor data;

modeling biobehavioral rhythms for each of said extracted specified sensor features to provide modeled biobehavioral rhythm data of the subject; determining rhythmicity characteristics of cyclical behavior of said modeled biobehavioral rhythm data of the subject; measuring stability of said determined rhythmicity characteristics of the subject across different time windows and/or across different populations to determine the deviation of the subject's rhythmicity characteristics from normal rhythmicity characteristics to predict health status and/or readiness status of the subject using a machine learning module; and transmitting said predication of health status and/or readiness status to a secondary source.

Example 2. The method of example 1, wherein said secondary source includes one or more of anyone of the following: local memory; remote memory; or display or graphical user interface.

Example 3. The method of example 1 (as well as subject matter in whole or in part of example 2), wherein said received sensor data comprises one or more of the following: behavioral signals or bio signals.

Example 4. The method of example 3, wherein said behavioral signals comprises one or more of the following: movement, audio, bluetooth, wifi, GPS, or logs of phone usage and communication.

Example 5. The method of example 3 (as well as subject matter in whole or in part of example 4), wherein said biosignals comprises one or more of the following: heart rate, skin temperature, or galvanic skin response.

Example 6. The method of example 1 (as well as subject matter of one or more of any combination of examples 2-5, in whole or in part), wherein health status includes one or more of the following: loneliness, depression, cancer, diabetes, or productivity.

Example 7. The method of example 1 (as well as subject matter of one or more of any combination of examples 2-6, in whole or in part), wherein said modeling of biobehavioral rhythms for each of said extracted specified sensor features applies to specified durations or periods.

Example 8. The method of example 1 (as well as subject matter of one or more of any combination of examples 2-7, in whole or in part), wherein said extracted specified sensor features are segmented into different windows of interest and sent to a rhythm discovery component that applies periodic functions on each windowed stream of said extracted specified sensor feature to detect their periodicity; and said detected periods are then used to model rhythmic function that represents the time series data stream for said extracted specified sensor feature, wherein said model rhythmic function includes parameters.

Example 9. The method of example 8, wherein: a) said parameters of said model rhythmic function are aggregated and further processed to characterize the stability or variation in rhythms; and b) said parameters of said model rhythmic function are used as features in said machine learning module for said predication of health status and/or readiness status of the subject.

Example 10. The method of example 8 (as well as subject matter in whole or in part of example 9), further comprising identifying rhythmicity in said time series data stream for detecting and observing cyclic behavior.

Example 11. The method of example 10, wherein said identification rhythmicity in said time series data stream is accomplished by applying an autocorrelation process or a periodogram process.

Example 12. The method of example 11, wherein said autocorrelation process includes an autocorrelation function (ACF) between two values yₜ, yₜ₋ₖ in a time series yₜ that is defined as

Corr(yₜ,yₜ₋ₖ),k=1,2, . . . ,

where k is the time gap and is called the lag.

Example 13. The method of example 11 (as well as subject matter in whole or in part of example 12), wherein said periodogram process provides a measure of strength and regularity of the underlying rhythm through estimation of the spectral density of a signal, wherein for a time series yₜ, t=1, 2, . . . , the spectral energy Pₖ of frequency k can be calculated as:

$${P}_{k} = {( \frac{2}{T} {\sum }_{t = 1}^{T} {y}_{t} cos ( \frac{2 π kt}{T} ) )}^{2} + {( \frac{2}{T} {\sum }_{t = 1}^{T} {y}_{t} sin ( \frac{2 π kt}{T} ) )}^{2} .$$

Example 14. The method of example 10 (as well as subject matter of one or more of any combination of examples 2-9 and 11-13, in whole or in part), further comprising modeling rhythmic behavior of said time series data, which is accomplished through a periodic function.

Example 15. The method of example 14, further comprising extracting rhythm parameters from the said modeling rhythmic behavior, wherein said rhythm parameters include one or more of the following: fundamental period, MESOR, magnitude, acrophase (PHI), orthophase, bathyphase, P-value (P), percent rhythm (PR), Integrated p-value (IP), integrated percent rhythm (IPR), or longest cycle of the model (LCM).

Example 16. The method of example 14 (as well as subject matter in whole or in part of example 15), wherein said modeling rhythmic behavior comprises modeling rhythms with known periods using Cosinor, wherein a cosine function to model said time series includes:

$${y}_{i} = M + {\sum }_{c = 1}^{C} {A}_{c} cos ( {ω}_{c} {t}_{i} + {ϕ}_{c} ) + {c}_{i} ,$$

where yᵢ is the observed value at time tᵢ; M presents the MESOR; tᵢ is the sampling time; C is the set of all periodic components; A_{c}, ω_{c}, ϕ_{c} respectively presents the amplitude, frequency, and acrophase of each periodic components; and eᵢ is the error term.

Example 17. The method of example 10 (as well as subject matter of one or more of any combination of examples 2-9 and 11-16, in whole or in part), further comprising using rhythm features of k consecutive time windows of said windows of interest and for a population of D data samples incorporates supervised and unsupervised machine learning methods.

Example 18. The method of example 17, wherein said supervised and unsupervised machine learning methods includes one of the following: regression, classification, or clustering process.

Example 19. The method of example 1 (as well as subject matter of one or more of any combination of examples 2-18, in whole or in part), wherein said measuring of stability is provided using an autocorrelation process and a Cosinor function process.

Example 20. A system configured for modeling biobehavioral rhythms of a subject. The system may comprise: a computer processor; and a memory configured to store instructions that are executable by the computer processor, wherein said processor is configured to execute the instructions to: receive sensor data collected from a mobile device and/or wearable device; extract specified sensor features from said received sensor data; model biobehavioral rhythms for each of said extracted specified sensor features to provide modeled biobehavioral rhythm data of the subject; determine rhythmicity characteristics of cyclical behavior of said modeled biobehavioral rhythm data of the subject; measure stability of said determined rhythmicity characteristics of the subject across different time windows and/or across different populations to determine the deviation of the subject's rhythmicity characteristics from normal rhythmicity characteristics to predict health status and/or readiness status of the subject using a machine learning module; and transmit said predication of health status and/or readiness status to a secondary source.

Example 21. The system of example 20, wherein said secondary source includes one or more of anyone of the following: local memory; remote memory; or display or graphical user interface.

Example 22. The system of example 20 (as well as subject matter in whole or in part of example 21), wherein said received sensor data comprises one or more of the following: behavioral signals or bio signals.

Example 23. The system of example 22, wherein said behavioral signals comprise one or more of the following: movement, audio, bluetooth, wifi, GPS, or logs of phone usage and communication.

Example 24. The system of example 22 (as well as subject matter in whole or in part of example 23), wherein said biosignal comprises one or more of the following: heart rate, skin temperature, or galvanic skin response.

Example 25. The system of example 20 (as well as subject matter of one or more of any combination of examples 21-24, in whole or in part), wherein health status includes one or more of the following: loneliness, depression, cancer, diabetes, or productivity.

Example 26. The system of example 20 (as well as subject matter of one or more of any combination of examples 21-25, in whole or in part), wherein said modeling of biobehavioral rhythms for each of said extracted specified sensor features applies to specified durations or periods.

Example 27. The system of example 20 (as well as subject matter of one or more of any combination of examples 21-26, in whole or in part), wherein said extracted specified sensor features are segmented into different windows of interest and sent to a rhythm discovery component that applies periodic functions on each windowed stream of said extracted specified sensor feature to detect their periodicity; and said detected periods are then used to model rhythmic function that represents the time series data stream for said extracted specified sensor feature, wherein said model rhythmic function includes parameters.

Example 28. The system of example 27, wherein: a) said parameters of said model rhythmic function are aggregated and further processed to characterize the stability or variation in rhythms; and b) said parameters of said model rhythmic function are used as features in said machine learning module for said predication of health status and/or readiness status of the subject.

Example 29. The system of example 27 (as well as subject matter in whole or in part of example 28), further comprising identifying rhythmicity in said time series data stream for detecting and observing cyclic behavior.

Example 30. The system of example 29, wherein said identification rhythmicity in said time series data stream is accomplished by applying an autocorrelation process or a periodogram process.

Example 31. The system of example 30, wherein said autocorrelation process includes an autocorrelation function (ACF) between two values yₜ, yₜ₋ₖ in a time series yₜ that is defined as

Corr(yₜ,yₜ₋ₖ),k=1,2, . . . ,

where k is the time gap and is called the lag.

Example 32. The system of example 30 (as well as subject matter in whole or in part of example 31), wherein said periodogram process provides a measure of strength and regularity of the underlying rhythm through estimation of the spectral density of a signal, wherein for a time series yₜ, t=1, 2, . . . , the spectral energy Pₖ of frequency k can be calculated as:

$${P}_{k} = {( \frac{2}{T} {\sum }_{t = 1}^{T} {y}_{t} cos ( \frac{2 π kt}{T} ) )}^{2} + {( \frac{2}{T} {\sum }_{t = 1}^{T} {y}_{t} sin ( \frac{2 π kt}{T} ) )}^{2} .$$

Example 33. The system of example 29 (as well as subject matter of one or more of any combination of examples 21-28 and 30-32, in whole or in part), further comprising modeling rhythmic behavior of said time series data, which is accomplished through a periodic function.

Example 34. The system of example 33, further comprising extracting rhythm parameters from the said modeling rhythmic behavior, wherein said rhythm parameters include one or more of the following: fundamental period, MESOR, magnitude, acrophase (PHI), orthophase, bathyphase, P-value (P), percent rhythm (PR), Integrated p-value (IP), integrated percent rhythm (IPR), or longest cycle of the model (LCM).

Example 35. The system of example 33 (as well as subject matter in whole or in part of example 34), wherein said modeling rhythmic behavior comprises modeling rhythms with known periods using Cosinor, wherein a cosine function to model said time series includes:

$${y}_{i} = M + {\sum }_{c = 1}^{C} {A}_{c} cos ( {ω}_{c} {t}_{i} + {ϕ}_{c} ) + {c}_{i} ,$$

where yᵢ is the observed value at time tᵢ; M presents the MESOR; ti is the sampling time; C is the set of all periodic components; A_{c}, ω_{c}, ϕ_{c} respectively presents the amplitude, frequency, and acrophase of each periodic components; and eᵢ is the error term.

Example 36. The system of example 29 (as well as subject matter of one or more of any combination of examples 21-28 and 30-35, in whole or in part), further comprising using rhythm features of k consecutive time windows of said windows of interest and for a population of D data samples incorporates supervised and unsupervised machine learning methods.

Example 37. The system of example 36, wherein said supervised and unsupervised machine learning methods includes one of the following: regression, classification, or clustering process.

Example 38. The system of example 20 (as well as subject matter of one or more of any combination of examples 21-37, in whole or in part), wherein said measuring of stability is provided using an autocorrelation process and a Cosinor function process.

Example 39. A computer program product, comprising a non-transitory computer-readable storage medium containing computer-executable instructions for modeling biobehavioral rhythms of a subject. The instructions causing the computer to: receive sensor data collected from a mobile device and/or wearable device; extract specified sensor features from said received sensor data; model biobehavioral rhythms for each of said extracted specified sensor features to provide modeled biobehavioral rhythm data of the subject; determine rhythmicity characteristics of cyclical behavior of said modeled biobehavioral rhythm data of the subject; measure stability of said determined rhythmicity characteristics of the subject across different time windows and/or across different populations to determine the deviation of the subject's rhythmicity characteristics from normal rhythmicity characteristics to predict health status and/or readiness status of the subject using a machine learning module; and transmit said predication of health status and/or readiness status to a secondary source.

Example 40. The computer program product of example 39, wherein said secondary source includes one or more of anyone of the following: local memory; remote memory; or display or graphical user interface.

Example 41. The computer program product of example 39 (as well as subject matter in whole or in part of example 40), wherein said received sensor data comprises one or more of the following: behavioral signals or biosignals.

Example 42. The computer program product of example 41, wherein said behavioral signals comprises one or more of the following: movement, audio, bluetooth, wifi, GPS, or logs of phone usage and communication.

Example 43. The computer program product of example 41 (as well as subject matter in whole or in part of example 42), wherein said biosignals comprises one or more of the following: heart rate, skin temperature, or galvanic skin response.

Example 44. The computer program product of example 39 (as well as subject matter of one or more of any combination of examples 40-43, in whole or in part), wherein health status includes one or more of the following: loneliness, depression, cancer, diabetes, or productivity.

Example 45. The computer program product of example 39 (as well as subject matter of one or more of any combination of examples 40-44, in whole or in part), wherein said modeling of biobehavioral rhythms for each of said extracted specified sensor features applies to specified durations or periods.

Example 46. The computer program product of example 39 (as well as subject matter of one or more of any combination of examples 40-45, in whole or in part), wherein said extracted specified sensor features are segmented into different windows of interest and sent to a rhythm discovery component that applies periodic functions on each windowed stream of said extracted specified sensor feature to detect their periodicity; and said detected periods are then used to model rhythmic function that represents the time series data stream for said extracted specified sensor feature, wherein said model rhythmic function includes parameters.

Example 47. The computer program product of example 46, wherein: a) said parameters of said model rhythmic function are aggregated and further processed to characterize the stability or variation in rhythms; and b) said parameters of said model rhythmic function are used as features in said machine learning module for said predication of health status and/or readiness status of the subject.

Example 48. The computer program product of example 46 (as well as subject matter in whole or in part of example 47), further comprising identifying rhythmicity in said time series data stream for detecting and observing cyclic behavior.

Example 49. The computer program product of example 48, wherein said identification rhythmicity in said time series data stream is accomplished by applying an autocorrelation process or a periodogram process.

Example 50. The computer program product of example 49, wherein said autocorrelation process includes an autocorrelation function (ACF) between two values yₜ, yₜ₋ₖ in a time series yₜ that is defined as

Corr(yₜ,yₜ₋ₖ),k=1,2, . . . ,

where k is the time gap and is called the lag.

Example 51. The computer program product of example 49 (as well as subject matter in whole or in part of example 50), wherein said periodogram process provides a measure of strength and regularity of the underlying rhythm through estimation of the spectral density of a signal, wherein for a time series yₜ, t=1, 2, . . . , T, the spectral energy Pₖ of frequency k can be calculated as:

$${P}_{k} = {( \frac{2}{T} {\sum }_{t = 1}^{T} {y}_{t} cos ( \frac{2 π kt}{T} ) )}^{2} + {( \frac{2}{T} {\sum }_{t = 1}^{T} {y}_{t} sin ( \frac{2 π kt}{T} ) )}^{2} .$$

Example 52. The computer program product of example 48 (as well as subject matter of one or more of any combination of examples 40-47 and 50-51, in whole or in part), further comprising modeling rhythmic behavior of said time series data, which is accomplished through a periodic function.

Example 53. The computer program product of example 52, further comprising extracting rhythm parameters from the said modeling rhythmic behavior, wherein said rhythm parameters include one or more of the following: fundamental period, MESOR, magnitude, acrophase (PHI), orthophase, bathyphase, P-value (P), percent rhythm (PR), Integrated p-value (IP), integrated percent rhythm (IPR), or longest cycle of the model (LCM).

Example 54. The computer program product of example 52 (as well as subject matter in whole or in part of example 53), wherein said modeling rhythmic behavior comprises modeling rhythms with known periods using Cosinor, wherein a cosine function to model said time series includes:

$${y}_{i} = M + {\sum }_{c = 1}^{C} {A}_{c} cos ( {ω}_{c} {t}_{i} + {ϕ}_{c} ) + {c}_{i} ,$$

where yᵢ is the observed value at time tᵢ; M presents the MESOR; ti is the sampling time; C is the set of all periodic components; A_{c}, ω_{c}, ϕ_{c} respectively presents the amplitude, frequency, and acrophase of each periodic components; and et is the error term.

Example 55. The computer program product of example 48 (as well as subject matter of one or more of any combination of examples 40-47 and 49-54, in whole or in part), further comprising using rhythm features of k consecutive time windows of said windows of interest and for a population of D data samples incorporates supervised and unsupervised machine learning methods.

Example 56. The computer program product of example 55, wherein said supervised and unsupervised machine learning methods includes one of the following: regression, classification, or clustering process.

Example 57. The computer program product of example 39 (as well as subject matter of one or more of any combination of examples 40-56, in whole or in part), wherein said measuring of stability is provided using an autocorrelation process and a Cosinor function process.

Example 58. A system configured to perform the method of any one or more of Examples 1-19.

Example 59. A computer program product configured to perform the method of any one or more of Examples 1-19.

Example 60. The method of using any of the elements, components, devices, computer program product and/or systems, or their sub-components, provided in any one or more of examples 20-38, in whole or in part.

Example 61. The method of manufacturing any of the elements, components, devices, computer program product and/or systems, or their sub-components, provided in any one or more of examples 20-38, in whole or in part.

### REFERENCES

The devices, systems, models, apparatuses, modules, compositions, computer program products, non-transitory computer readable medium, models, algorithms, and methods of various embodiments of the invention disclosed herein may utilize aspects (devices, systems, models, apparatuses, modules, compositions, computer program products, non-transitory computer readable medium, models, algorithms, and methods) disclosed in the following references, applications, publications and patents and which are hereby incorporated by reference herein in their entirety, and which are not admitted to be prior art with respect to the present invention by inclusion in this section:
[1] Saeed Abdullah, Mark Matthews, Elizabeth L Murnane, Geri Gay, and Tanzeem Choudhury. 2014. Towards circadian computing: “early to bed and early to rise” makes some of us unhealthy and sleep deprived. In Proceedings of the 2014 ACM international joint conference on pervasive and ubiquitous computing. 673-684.
[2] Saeed Abdullah, Elizabeth L Murnane, Mark Matthews, and Tanzeem Choudhury. 2017. Circadian computing: sensing, modeling, and maintaining biological rhythms. In Mobile health. Springer, 35-58.
[3] Saeed Abdullah, Elizabeth L Murnane, Mark Matthews, Matthew Kay, Julie A Kientz, Geri Gay, and Tanzeem Choudhury. 2016. Cognitive rhythms: unobtrusive and continuous sensing of alertness using a mobile phone. In Proceedings of the 2016 ACM International Joint Conference on Pervasive and Ubiquitous Computing. 178-189.
[4] Aaron T Beck, Robert A Steer, Roberta Ball, and William F Ranieri. 1996. Comparison of Beck Depression Inventories-IA and-II in psychiatric outpatients. Journal of personality assessment 67, 3 (1996), 588-597.
[5] Giannina J Bellone, Santiago A Plano, Daniel P Cardinali, Daniel Perez Chada, Daniel E Vigo, and Diego A Golombek. 2016. Comparative analysis of actigraphy performance in healthy young subjects. Sleep Science 9, 4 (2016), 272-279.
[6] Peter Bloomfield. 2004. Fourier analysis of time series: an introduction. John Wiley & Sons.
[7] Amy M Bohnert, Julie Wargo Aikins, and Nicole T Arola. 2013. Regrouping: Organized activity involvement and social adjustment across the transition to high school. New directions for child and adolescent development 2013, 140 (2013), 57-75.
[8] Michael J Bradburn, Jonathan J Deeks, Jesse A Berlin, and A Russell Localio. 2007. Much ado about nothing: a comparison of the performance of meta-analytical methods with rare events. Statistics in medicine 26, 1 (2007), 53-77.
[9] Leo Breiman. 2001. Random forests. Machine learning 45, 1 (2001), 5-32.
[10] Lars Buitinck, Gilles Louppe, Mathieu Blondel, Fabian Pedregosa, Andreas Mueller, Olivier Grisel, Vlad Niculae, Peter Prettenhofer, Alexandre Gramfort, Jaques Grobler, Robert Layton, Jake VanderPlas, Arnaud Joly, Brian Holt, and Gael Varoquaux. 2013. API design for machine learning software: experiences from the scikit-learn project. In ECML PKDD Workshop: Languages for Data Mining and Machine Learning. 108-122.
[11] John Parker Burg. 1972. The relationship between maximum entropy spectra and maximum likelihood spectra. Geophysics 37, 2 (1972), 375-376.
[12] Germaine Cornélissen. 2014. Cosinor-based rhythmometry. Theoretical biology medical modelling 11 (04 2014), 16. https://doi.org/10.1186/1742-4682-11-16
[13] Maria J Costa, Barbel Finkenstädt, Véronique Roche, Francis Lévi, Peter D Gould, Julia Foreman, Karen Halliday, Anthony Hall, and David A Rand. 2013. Inference on periodicity of circadian time series. Biostatistics 14, 4 (2013), 792-806.
[14] Pietro Cugini. 1993. Chronobiology: principles and methods. ANNALI-ISTITUTO SUPERIORE DI SANITA 29 (1993), 483-483.
[15] TG Dietterich. 2000. Ensemble methods in machine learning. Multiple Classifier Systems: First International Workshop, MCS 2000, Lecture Notes in Computer Science, 1-15.
[16] Yipeng Ding and Jingtian Tang. 2014. Micro-Doppler trajectory estimation of pedestrians using a continuous-wave radar. IEEE Transactions on Geoscience and Remote Sensing 52, 9 (2014), 5807-5819.
[17] Afsaneh Doryab, Prerna Chikarsel, Xinwen Liu, and Anind Day. 2018. Extraction of Behavioral Features from Smartphone and Wearable Data. arXiv, Jan. 9, 2019. http://arxiv.org/abs/1812.10394.
[18] Afsaneh Doryab, Anind K. Dey, Grace Kao, and Carissa Low. 2019. Modeling Biobehavioral Rhythms with Passive Sensing in the Wild: A Case Study to Predict Readmission Risk after Pancreatic Surgery. Proc. ACM Interact. Mob. Wearable Ubiquitous Technol. 3, 1, Article 8 (March 2019), 21 pages. https://doi.org/10.1145/3314395
[19] Afsaneh Doryab, Daniella K Villalba, Prerna Chikersal, Janine M Dutcher, Michael Tumminia, Xinwen Liu, Sheldon Cohen, Kasey Creswell, Jennifer Mankoff, John D Creswell, et al. 2019. Identifying Behavioral Phenotypes of Loneliness and Social Isolation with Passive Sensing: Statistical Analysis, Data Mining and Machine Learning of Smartphone and Fitbit Data. JMIR mHealth and uHealth 7, 7 (2019), e13209.
[20] Harold B. Dowse. 2009. Chapter 6 Analyses for Physiological and Behavioral Rhythmicity. In Computer Methods, Part A. Methods in Enzymology, Vol. 454. Academic Press, 141-174. https://doi.org/10.1016/S0076-6879 (08)03806-8.
[21] David J A Dozois, Keith S Dobson, and Jamie L Ahnberg. 1998. A psychometric evaluation of the Beck Depression Inventory—II. Psychological assessment 10, 2 (1998), 83.
[22] Kieron D Edwards, Ozgur E Akman, Kirsten Knox, Peter J Lumsden, Adrian W Thomson, Paul E Brown, Alexandra Pokhilko, Laszlo Kozma-Bognar, Ferenc Nagy, David A Rand, et al. 2010. Quantitative analysis of regulatory flexibility under changing environmental conditions. Molecular systems biology 6, 1 (2010).
[23] JT Enright. 1965. The search for rhythmicity in biological time-series. Journal of theoretical Biology 8, 3 (1965), 426-468.
[24] JR Fernández, RC Hermida, and A Mojón. 2009. Chronobiological analysis techniques. Application to blood pressure. Philosophical Transactions of the Royal Society A: Mathematical, Physical and Engineering Sciences 367, 1887 (2009), 431-445.
[25] Denzil Ferreira, Vassilis Kostakos, and Anind Dey. 2015. AWARE: Mobile Context Instrumentation Framework. Frontiers in ICT 2 (05 2015). https://doi.org/10.3389/fict.2015.00006
[26] Jerome Friedman. 2001. Greedy Function Approximation: A Gradient Boosting Machine. Annals of Statistics 29 (10 2001), 1189-1232. https://doi.org/10.2307/2699986
[27] John Gale, Heather Cox, Jingyi Qian, Gene Block, Christopher Colwell, and Aleksey Matveyenko. 2011. Disruption of Circadian Rhythms Accelerates Development of Diabetes through Pancreatic Beta-Cell Loss and Dysfunction. Journal of biological rhythms 26 (10 2011), 423-33. https://doi.org/10.1177/0748730411416341
[28] Quentin Geissmann, Luis Garcia Rodriguez, Esteban J Beckwith, and Giorgio F Gilestro. 2019. Rethomics: An R framework to analyse high-throughput behavioural data. PloS one 14, 1 (2019).
[29] Anne Germain and David Kupfer. 2008. Circadian rhythm disturbances in depression. Human psychopharmacology 23 (10 2008), 571-85. https://doi.org/10.1002/hup.964
[30] M Gleicher, T Landesberger von Antburg, and I Viola. [n.d.]. ARGUS: An Interactive Visual Analytics Framework For the Discovery of Disruptions in Bio-Behavioral Rhythms. ([n. d.]).
[31] Franz Halberg. 1969. Chronobiology. Annual review of physiology 31, 1 (1969), 675-726.
[32] Johnni Hansen. 2017. Night shift work and risk of breast cancer. Current environmental health reports 4, 3 (2017), 325-339.
[33] Elizabeth Klerman, Andrew Phillips, and Matt Bianchi. 2016. Statistics for Sleep and Biological Rhythms Research: Longitudinal Analysis of Biological Rhythms Data. Journal of Biological Rhythms 32 (10 2016). https://doi.org/10.1177/0748730416670051
[34] Fulton Koehler, F K Okano, Lila R. Elveback, Franz Halberg, and John J. Bittner. 1956. Periodograms for the study of physiologic daily periodicity in mice and in man; with a procedural outline and some tables for their computation. Experimental medicine and surgery 14 1 (1956), 5-30.
[35] Alexander Kraskov, Harald Stogbauer, and Peter Grassberger. 2004. Estimating mutual information. Phys. Rev. E 69 (June 2004), 066138. Issue 6. https://doi.org/10.1103/PhysRevE.69.066138
[36] Gloria Kuhn. 2001. Circadian rhythm, shift work, and emergency medicine. Annals of emergency medicine 37, 1 (2001), 88-98.
[37] Hyun-Ah Lee, Heon-Jeong Lee, Joung-Ho Moon, Taek Lee, Min-Gwan Kim, Hoh In, Chul-Hyun Cho, and Leen Kim. 2017. Comparison of wearable activity tracker with actigraphy for sleep evaluation and circadian rest-activity rhythm measurement in healthy young adults. Psychiatry investigation 14, 2 (2017), 179.
[38] Cathy Lee Gierke and Germaine Cornelissen. 2016. Chronomics analysis toolkit (CATkit). Biological Rhythm Research 47, 2 (2016), 163-181.
[39] Nicholas R Lomb. 1976. Least-squares frequency analysis of unequally spaced data. Astrophysics and space science 39, 2 (1976), 447-462.
[40] Anmol Madan, Manuel Cebrián, David Lazer, and Alex Pentland. 2010. Social Sensing for Epidemiological Behavior Change. UbiComp'10—Proceedings of the 2010 ACM Conference on Ubiquitous Computing, 291-300. https://doi.org/10.1145/1864349.1864394
[41] Janna Mantua, Nickolas Gravel, and Rebecca Spencer. 2016. Reliability of sleep measures from four personal health monitoring devices compared to research-based actigraphy and polysomnography. Sensors 16, 5 (2016), 646.
[42] Nicolai Meinshausen and Peter Balmann. 2010. Stability selection. Journal of the Royal Statistical Society: Series B (Statistical Methodology) 72, 4 (2010), 417-473. https://doi.org/10.1111/j.1467-9868.2010.00740.x arXiv: https://rss.onlinelibrary.wiley.com/doi/pdf/10.1111/j.1467-9868.2010.00740.x
[43] Scott Menard. 2002. Applied logistic regression analysis. Vol. 106. Sage.
[44] Jun-Ki Min, Afsaneh Doryab, Jason Wiese, Shahriyar Amini, John Zimmerman, and Jason I Hong. 2014. Toss'n'turn: smartphone as sleep and sleep quality detector. In Proceedings of the SIGCHI conference on human factors in computing systems. 477-486.
[45] Marie-Christine Mormont, James Waterhouse, Pascal Bleuzen, Sylvie Giacchetti, Alain Jami, André Bogdan, Joseph Lellouch, Jean-Louis Misset, Yvan Touitou, and Francis Lévi. 2000. Marked 24-h rest/activity rhythms are associated with better quality of life, better response, and longer survival in patients with metastatic colorectal cancer and good performance status. Clinical Cancer Research 6, 8 (2000), 3038-3045.
[46] Elizabeth L Murnane, Saeed Abdullah, Mark Matthews, Tanzeem Choudhury, and Geri Gay. 2015. Social (media) jet lag: How usage of social technology can modulate and reflect circadian rhythms. In Proceedings of the 2015 ACM International Joint Conference on Pervasive and Ubiquitous Computing. 843-854.
[47] Erika Nelson-Wong, Sam Howarth, David A Winter, and Jack P Callaghan. 2009. Application of autocorrelation and cross-correlation analyses in human movement and rehabilitation research. journal of orthopaedic & sports physical therapy 39, 4 (2009), 287-295.
[48] M Poyurovsky, R Nave, R Epstein, O Tzischinsky, M Schneidman, T R E Barnes, A Weizman, and P Lavie. 2000. Actigraphic monitoring (actigraphy) of circadian locomotor activity in schizophrenic patients with acute neuroleptic-induced akathisia. European Neuropsychopharmacology 10, 3 (2000), 171-176.
[49] Jonathon Pye, Andrew J K Phillips, SeanWCain, Maryam Montazerolghaem, Loren Mowszowski, Shantel Duffy, Ian B Hickie, and Sharon L Naismith. 2021. Irregular sleep-wake patterns in older adults with current or remitted depression. Journal of Affective Disorders 281 (2021), 431-437.
[50] Roberto Refinetti, Germaine Cornélissen, and Franz Halberg. 2007. Procedures for numerical analysis of circadian rhythms. Biological rhythm research 38, 4 (2007), 275-325.
[51] Roberto Refinetti and Michael Menaker. 1992. The circadian rhythm of body temperature. Physiology & behavior 51, 3 (1992), 613-637.
[52] Alain Reinberg and Israel Ashkenazi. 2003. Concepts in human biological rhythms. Dialogues in clinical neuroscience 5, 4 (2003), 327.
[53] Daniel W Russell. 1996. UCLA Loneliness Scale (Version 3): Reliability, validity, and factor structure. Journal of personality assessment 66, 1 (1996), 20-40.
[54] Sohrab Saeb, Mi Zhang, Christopher Karr, Stephen Schueller, Marya Corden, Konrad Kording, and David Mohr. 2015. Mobile Phone Sensor Correlates of Depressive Symptom Severity in Daily-Life Behavior: An Exploratory Study. Journal of Medical Internet Research 17 (07 2015). https://doi.org/10.2196/jmir.4273
[55] Jeffrey Scargle. 1989. Studies in astronomical time series analysis. III. Fourier transforms, autocorrelation functions, and cross-correlation functions of unevenly spaced data. Astrophysical Journal 343 (08 1989). https://doi.org/10.1086/167757
[56] Arthur Schuster. 1898. On the investigation of hidden periodicities with application to a supposed 26 day period of meteorological phenomena. Terrestrial Magnetism 3, 1 (1898), 13-41. https://doi.org/10.1029/TM003i001p00013 arXiv: https://agupubs.onlinelibrary.wiley.com/doi/pdf/10.1029/TM003i001p00013
[57] Phillip G Sokolove and Wayne N Bushell. 1978. The chi square periodogram: its utility for analysis of circadian rhythms. Journal of theoretical biology 72, 1 (1978), 131-160.
[58] Martin Straume, Susan G Frasier-Cadoret, and Michael L Johnson. 2002. Least-squares analysis of fluorescence data. In Topics in fluorescence spectroscopy. Springer, 177-240.
[59] Gunilla Brun Sundblad, Anna Jansson, Tönu Saartok, Per Renström, and Lars-Magnus Engström. 2008. Self-rated pain and perceived health in relation to stress and physical activity among school-students: A 3-year follow-up. Pain 136, 3 (2008), 239-249.
[60] John M. Taub. 1978. Behavioral and psychophysiological correlates of irregularity in chronic sleep routines. Biological Psychology 7, 1 (1978), 37-53. https://doi.org/10.1016/0301-0511 (78)90041-8
[61] Rui Wang, Fanglin Chen, Zhenyu Chen, Tianxing Li, Gabriella Harari, Stefanie Tignor, Xia Zhou, Dror Ben-Zeev, and Andrew Campbell. 2014. StudentLife: Assessing mental health, academic performance and behavioral trends of college students using smartphones. UbiComp 2014-Proceedings of the 2014 ACM International Joint Conference on Pervasive and Ubiquitous Computing. https://doi.org/10.1145/2632048.2632054
[62] Gregory William Yeutter. 2016. Determination of Circadian Rhythms in Consumer-Grade Actigraphy Devices. Drexel University.
[63] Ping Zhang. 1993. Model selection via multifold cross validation. The Annals of Statistics (1993), 299-313.
[64] Tomasz Zielinski, Anne M Moore, Eilidh Troup, Karen J Halliday, and Andrew J Millar. 2014. Strengths and limitations of period estimation methods for circadian data. PloS one 9, 5 (2014).
[65]. Trovato B and Tobin G K M, et al., “Productivity Prediction via Biobehavioral Rhythms Modeled from Multimodal Mobile Data Streams: A Feasibility Study”, Woodstock '18, Jun. 3-5, 2018, Woodstock, N.Y.; 1-10. https://doi.org/10.1145/1122445.1122456.
[66]. Runze and Xinwen, et al., “Similarity Measurement of Cyclic Multimodal Mobile Timeseries Data with Case Studies in Biobehavioral Rhythms”, Woodstock '18, June 3-5, 2018, Woodstock, N.Y. https://doi.org/10.1145/1122445.1122456

In summary, while the present invention has been described with respect to specific embodiments, many modifications, variations, alterations, substitutions, and equivalents will be apparent to those skilled in the art. The present invention is not to be limited in scope by the specific embodiment described herein. Indeed, various modifications of the present invention, in addition to those described herein, will be apparent to those of skill in the art from the foregoing description and accompanying drawings. Accordingly, the invention is to be considered as limited only by the spirit and scope of the following claims including all modifications and equivalents.

Still other embodiments will become readily apparent to those skilled in this art from reading the above-recited detailed description and drawings of certain exemplary embodiments. It should be understood that numerous variations, modifications, and additional embodiments are possible, and accordingly, all such variations, modifications, and embodiments are to be regarded as being within the spirit and scope of this application. For example, regardless of the content of any portion (e.g., title, field, background, summary, abstract, drawing figure, etc.) of this application, unless clearly specified to the contrary, there is no requirement for the inclusion in any claim herein or of any application claiming priority hereto of any particular described or illustrated activity or element, any particular sequence of such activities, or any particular interrelationship of such elements. Moreover, any activity can be repeated, any activity can be performed by multiple entities, and/or any element can be duplicated. Further, any activity or element can be excluded, the sequence of activities can vary, and/or the interrelationship of elements can vary. Unless clearly specified to the contrary, there is no requirement for any particular described or illustrated activity or element, any particular sequence or such activities, any particular size, speed, material, dimension or frequency, or any particularly interrelationship of such elements. Accordingly, the descriptions and drawings are to be regarded as illustrative in nature, and not as restrictive. Moreover, when any number or range is described herein, unless clearly stated otherwise, that number or range is approximate. When any range is described herein, unless clearly stated otherwise, that range includes all values therein and all sub ranges therein. Any information in any material (e.g., a United States/foreign patent, United States/foreign patent application, book, article, etc.) that has been incorporated by reference herein, is only incorporated by reference to the extent that no conflict exists between such information and the other statements and drawings set forth herein. In the event of such conflict, including a conflict that would render invalid any claim herein or seeking priority hereto, then any such conflicting information in such incorporated by reference material is specifically not incorporated by reference herein.

What is claimed is:

1. A computer-implemented method for modeling biobehavioral rhythms of a subject, said method comprising:
receiving sensor data collected from a mobile device and/or wearable device;
extracting specified sensor features from said received sensor data;
modeling biobehavioral rhythms for each of said extracted specified sensor features to provide modeled biobehavioral rhythm data of the subject;
determining rhythmicity characteristics of cyclical behavior of said modeled biobehavioral rhythm data of the subject;
measuring stability of said determined rhythmicity characteristics of the subject across different time windows and/or across different populations to determine the deviation of the subject's rhythmicity characteristics from normal rhythmicity characteristics to predict health status and/or readiness status of the subject using a machine learning module; and
transmitting said predication of health status and/or readiness status to a secondary source.

2. The method of claim 1, wherein said secondary source includes one or more of anyone of the following:
local memory;
remote memory; or
display or graphical user interface.

3. The method of claim 1, wherein said received sensor data comprises one or more of the following:
behavioral signals or bio signals.

4. The method of claim 3, wherein said behavioral signals comprises one or more of the following:
movement, audio, bluetooth, wifi, GPS, or logs of phone usage and communication.

5. The method of claim 3, wherein said biosignals comprises one or more of the following:
heart rate, skin temperature, or galvanic skin response.

6. The method of claim 1, wherein health status includes one or more of the following: loneliness, depression, cancer, diabetes, or productivity.

7. The method of claim 1, wherein said modeling of biobehavioral rhythms for each of said extracted specified sensor features applies to specified durations or periods.

8. The method of claim 1, wherein said extracted specified sensor features are segmented into different windows of interest and sent to a rhythm discovery component that applies periodic functions on each windowed stream of said extracted specified sensor feature to detect their periodicity; and
said detected periods are then used to model rhythmic function that represents the time series data stream for said extracted specified sensor feature, wherein said model rhythmic function includes parameters.

9. The method of claim 8, wherein:
a) said parameters of said model rhythmic function are aggregated and further processed to characterize the stability or variation in rhythms; and
b) said parameters of said model rhythmic function are used as features in said machine learning module for said predication of health status and/or readiness status of the subject.

10. The method of claim 8, further comprising identifying rhythmicity in said time series data stream for detecting and observing cyclic behavior.

11. The method of claim 10, wherein said identification rhythmicity in said time series data stream is accomplished by applying an autocorrelation process or a periodogram process.

12. The method of claim 11, wherein said autocorrelation process includes an autocorrelation function (ACF) between two values yₜ, yₜ₋ₖ in a time series yₜ that is defined as
Corr(yₜ,yₜ₋ₖ),k=1,2, . . . ,
where k is the time gap and is called the lag.

13. The method of claim 11, wherein said periodogram process provides a measure of strength and regularity of the underlying rhythm through estimation of the spectral density of a signal, wherein for a time series yₜ, t=1, 2, . . . , the spectral energy Pₖ of frequency k can be calculated as:
$${P}_{k} = {( \frac{2}{T} {\sum }_{t = 1}^{T} {y}_{t} cos ( \frac{2 π kt}{T} ) )}^{2} + {( \frac{2}{T} {\sum }_{t = 1}^{T} {y}_{t} sin ( \frac{2 π kt}{T} ) )}^{2} .$$

14. The method of claim 10, further comprising modeling rhythmic behavior of said time series data, which is accomplished through a periodic function.

15. The method of claim 14, further comprising extracting rhythm parameters from the said modeling rhythmic behavior, wherein said rhythm parameters include one or more of the following:
fundamental period, MESOR, magnitude, acrophase (PHI), orthophase, bathyphase, P-value (P), percent rhythm (PR), Integrated p-value (IP), integrated percent rhythm (IPR), or longest cycle of the model (LCM).

16. The method of claim 14, wherein said modeling rhythmic behavior comprises modeling rhythms with known periods using Cosinor, wherein a cosine function to model said time series includes:
$${y}_{i} = M + {\sum }_{c = 1}^{C} {A}_{c} cos ( {ω}_{c} {t}_{i} + {ϕ}_{c} ) + {c}_{i} ,$$
where yᵢ is the observed value at time tᵢ; M presents the MESOR; ti is the sampling time; C is the set of all periodic components; A_{c}, ω_{c}, ϕ_{c} respectively presents the amplitude, frequency, and acrophase of each periodic components; and eᵢ is the error term.

17. The method of claim 10, further comprising using rhythm features of k consecutive time windows of said windows of interest and for a population of D data samples incorporates supervised and unsupervised machine learning methods.

18. The method of claim 17, wherein said supervised and unsupervised machine learning methods includes one of the following: regression, classification, or clustering process.

19. The method of claim 1, wherein said measuring of stability is provided using an autocorrelation process and a Cosinor function process.

20. A system configured for modeling biobehavioral rhythms of a subject, said system comprising:
a computer processor; and
a memory configured to store instructions that are executable by the computer processor, wherein said processor is configured to execute the instructions to:
receive sensor data collected from a mobile device and/or wearable device;
extract specified sensor features from said received sensor data;
model biobehavioral rhythms for each of said extracted specified sensor features to provide modeled biobehavioral rhythm data of the subject;
determine rhythmicity characteristics of cyclical behavior of said modeled biobehavioral rhythm data of the subject;
measure stability of said determined rhythmicity characteristics of the subject across different time windows and/or across different populations to determine the deviation of the subject's rhythmicity characteristics from normal rhythmicity characteristics to predict health status and/or readiness status of the subject using a machine learning module; and
transmit said predication of health status and/or readiness status to a secondary source.

21. The system of claim 20, wherein said secondary source includes one or more of anyone of the following:
local memory;
remote memory; or
display or graphical user interface.

22. The system of claim 20, wherein said received sensor data comprises one or more of the following:
behavioral signals or bio signals.

23. The system of claim 22, wherein said behavioral signals comprise one or more of the following:
movement, audio, bluetooth, wifi, GPS, or logs of phone usage and communication.

24. The system of claim 22, wherein said biosignal comprises one or more of the following:
heart rate, skin temperature, or galvanic skin response.

25. The system of claim 20, wherein health status includes one or more of the following: loneliness, depression, cancer, diabetes, or productivity.

26. The system of claim 20, wherein said modeling of biobehavioral rhythms for each of said extracted specified sensor features applies to specified durations or periods.

27. The system of claim 20, wherein said extracted specified sensor features are segmented into different windows of interest and sent to a rhythm discovery component that applies periodic functions on each windowed stream of said extracted specified sensor feature to detect their periodicity; and
said detected periods are then used to model rhythmic function that represents the time series data stream for said extracted specified sensor feature, wherein said model rhythmic function includes parameters.

28. The system of claim 27, wherein:
a) said parameters of said model rhythmic function are aggregated and further processed to characterize the stability or variation in rhythms; and
b) said parameters of said model rhythmic function are used as features in said machine learning module for said predication of health status and/or readiness status of the subject.

29. The system of claim 27, further comprising identifying rhythmicity in said time series data stream for detecting and observing cyclic behavior.

30. The system of claim 29, wherein said identification rhythmicity in said time series data stream is accomplished by applying an autocorrelation process or a periodogram process.

31. The system of claim 30, wherein said autocorrelation process includes an autocorrelation function (ACF) between two values yₜ, yₜ₋ₖ in a time series yₜ that is defined as
Corr(yₜ,yₜ₋ₖ),k=1,2, . . . ,
where k is the time gap and is called the lag.

32. The system of claim 30, wherein said periodogram process provides a measure of strength and regularity of the underlying rhythm through estimation of the spectral density of a signal, wherein for a time series yₜ, t=1, 2, . . . , the spectral energy Pₖ of frequency k can be calculated as:
$${P}_{k} = {( \frac{2}{T} {\sum }_{t = 1}^{T} {y}_{t} cos ( \frac{2 π kt}{T} ) )}^{2} + {( \frac{2}{T} {\sum }_{t = 1}^{T} {y}_{t} sin ( \frac{2 π kt}{T} ) )}^{2} .$$

33. The system of claim 29, further comprising modeling rhythmic behavior of said time series data, which is accomplished through a periodic function.

34. The system of claim 33, further comprising extracting rhythm parameters from the said modeling rhythmic behavior, wherein said rhythm parameters include one or more of the following:
fundamental period, MESOR, magnitude, acrophase (PHI), orthophase, bathyphase, P-value (P), percent rhythm (PR), Integrated p-value (IP), integrated percent rhythm (IPR), or longest cycle of the model (LCM).

35. The system of claim 33, wherein said modeling rhythmic behavior comprises modeling rhythms with known periods using Cosinor, wherein a cosine function to model said time series includes:
$${y}_{i} = M + {\sum }_{c = 1}^{C} {A}_{c} cos ( {ω}_{c} {t}_{i} + {ϕ}_{c} ) + {c}_{i} ,$$
where yᵢ is the observed value at time tᵢ; M presents the MESOR; ti is the sampling time; C is the set of all periodic components; A_{c}, ω_{c}, ϕ_{c} respectively presents the amplitude, frequency, and acrophase of each periodic components; and eᵢ is the error term.

36. The system of claim 29, further comprising using rhythm features of k consecutive time windows of said windows of interest and for a population of D data samples incorporates supervised and unsupervised machine learning methods.

37. The system of claim 36, wherein said supervised and unsupervised machine learning methods includes one of the following: regression, classification, or clustering process.

38. The system of claim 20, wherein said measuring of stability is provided using an autocorrelation process and a Cosinor function process.

39. A computer program product, comprising a non-transitory computer-readable storage medium containing computer-executable instructions for modeling biobehavioral rhythms of a subject, said instructions causing the computer to:
receive sensor data collected from a mobile device and/or wearable device;
extract specified sensor features from said received sensor data;
model biobehavioral rhythms for each of said extracted specified sensor features to provide modeled biobehavioral rhythm data of the subject;
determine rhythmicity characteristics of cyclical behavior of said modeled biobehavioral rhythm data of the subject;
measure stability of said determined rhythmicity characteristics of the subject across different time windows and/or across different populations to determine the deviation of the subject's rhythmicity characteristics from normal rhythmicity characteristics to predict health status and/or readiness status of the subject using a machine learning module; and
transmit said predication of health status and/or readiness status to a secondary source.

40. The computer program product of claim 39, wherein said secondary source includes one or more of anyone of the following:
local memory;
remote memory; or
display or graphical user interface.

41. The computer program product of claim 39, wherein said received sensor data comprises one or more of the following:
behavioral signals or bio signals.

42. The computer program product of claim 41, wherein said behavioral signals comprises one or more of the following:
movement, audio, bluetooth, wifi, GPS, or logs of phone usage and communication.

43. The computer program product of claim 41, wherein said biosignals comprises one or more of the following:
heart rate, skin temperature, or galvanic skin response.

44. The computer program product of claim 39, wherein health status includes one or more of the following: loneliness, depression, cancer, diabetes, or productivity.

45. The computer program product of claim 39, wherein said modeling of biobehavioral rhythms for each of said extracted specified sensor features applies to specified durations or periods.

46. The computer program product of claim 39, wherein said extracted specified sensor features are segmented into different windows of interest and sent to a rhythm discovery component that applies periodic functions on each windowed stream of said extracted specified sensor feature to detect their periodicity; and
said detected periods are then used to model rhythmic function that represents the time series data stream for said extracted specified sensor feature, wherein said model rhythmic function includes parameters.

47. The computer program product of claim 46, wherein:
a) said parameters of said model rhythmic function are aggregated and further processed to characterize the stability or variation in rhythms; and
b) said parameters of said model rhythmic function are used as features in said machine learning module for said predication of health status and/or readiness status of the subject.

48. The computer program product of claim 46, further comprising identifying rhythmicity in said time series data stream for detecting and observing cyclic behavior.

49. The computer program product of claim 48, wherein said identification rhythmicity in said time series data stream is accomplished by applying an autocorrelation process or a periodogram process.

50. The computer program product of claim 49, wherein said autocorrelation process includes an autocorrelation function (ACF) between two values yₜ, yₜ₋ₖ in a time series yₜ that is defined as
Corr(yₜ,yₜ₋ₖ),k=1,2, . . . ,
where k is the time gap and is called the lag.

51. The computer program product of claim 49, wherein said periodogram process provides a measure of strength and regularity of the underlying rhythm through estimation of the spectral density of a signal, wherein for a time series yₜ, t=1, 2, . . . , T, the spectral energy Pₖ of frequency k can be calculated as:
$${P}_{k} = {( \frac{2}{T} {\sum }_{t = 1}^{T} {y}_{t} cos ( \frac{2 π kt}{T} ) )}^{2} + {( \frac{2}{T} {\sum }_{t = 1}^{T} {y}_{t} sin ( \frac{2 π kt}{T} ) )}^{2} .$$

52. The computer program product of claim 48, further comprising modeling rhythmic behavior of said time series data, which is accomplished through a periodic function.

53. The computer program product of claim 52, further comprising extracting rhythm parameters from the said modeling rhythmic behavior, wherein said rhythm parameters include one or more of the following:
fundamental period, MESOR, magnitude, acrophase (PHI), orthophase, bathyphase, P-value (P), percent rhythm (PR), Integrated p-value (IP), integrated percent rhythm (IPR), or longest cycle of the model (LCM).

54. The computer program product of claim 52, wherein said modeling rhythmic behavior comprises modeling rhythms with known periods using Cosinor, wherein a cosine function to model said time series includes:
$${y}_{i} = M + {\sum }_{c = 1}^{C} {A}_{c} cos ( {ω}_{c} {t}_{i} + {ϕ}_{c} ) + {c}_{i} ,$$
where yᵢ is the observed value at time tᵢ; M presents the MESOR; ti is the sampling time; C is the set of all periodic components; A_{c}, ω_{c}, ϕ_{c} respectively presents the amplitude, frequency, and acrophase of each periodic components; and eᵢ is the error term.

55. The computer program product of claim 48, further comprising using rhythm features of k consecutive time windows of said windows of interest and for a population of D data samples incorporates supervised and unsupervised machine learning methods.

56. The computer program product of claim 55, wherein said supervised and unsupervised machine learning methods includes one of the following: regression, classification, or clustering process.

57. The computer program product of claim 39, wherein said measuring of stability is provided using an autocorrelation process and a Cosinor function process.
